(19) United States
(12) Patent Application Publication
(10) Pub. No.: US 2024/0344105 A1
(43) Pub. Date: Oct. 17, 2024

(54) METHODS FOR DETERMINING RISK OF MORTALITY FROM COVID-19

(71) Applicant: The University of Chicago, Chicago, IL (US)

(72) Inventors: Matthew R. Stutz, Chicago, IL (US); Nick Dylla, Chicago, IL (US); Eric G. Pamer, Chicago, IL (US)

(21) Appl. No.: 18/448,454

(22) Filed: Aug. 11, 2023

Related U.S. Application Data
(60) Provisional application No. 63/397,103, filed on Aug. 11, 2022.

(51) Int. Cl. C12Q 1/06; C12Q 1/10

(52) U.S. Cl. CPC C12Q 1/06; C12Q 1/10; G01N 2333/195; G01N 2333/24; G01N 2800/26; G01N 2800/52

(57) ABSTRACT

Provided herein are biomarkers associated with mortality in COVID-19 patients. In particular, provided herein are fecal biomarkers that correlate with elevated risk of mortality in subjects suffering from COVID-19 with severe respiratory insufficiency, and methods of treating COVID-19 based thereon.

### CROSS-REFERENCE TO RELATED APPLICATION

The present invention claims the benefit of U.S. Provisional Patent Application Ser. No. 63/397,103, filed Aug. 11, 2022, which is hereby incorporated by reference in its entirety.

### FIELD

Provided herein are biomarkers associated with mortality in COVID-19 patients. In particular, provided herein are fecal biomarkers that correlate with elevated risk of mortality in subjects suffering from COVID-19 with severe respiratory insufficiency, and methods of treating COVID-19 based thereon.

### BACKGROUND

SARS-CoV-2, the cause of COVID-19, has infected nearly 500 million individuals, leading to over 6 million deaths worldwide as of April 2022. Mortality from infection can occur within days after symptoms develop or many weeks later. Respiratory failure and death during early infection are associated with high pulmonary SARS-CoV-2 titers, epithelial and endothelial injury, infection of epithelial basal cells, neutrophil infiltration and induction of interferon stimulated genes (ISGs) while late mortality is associated with pulmonary infiltration with CD8 T cells expressing PD1, activated macrophages and reduced ISG transcript levels (refs. 1-2; incorporated by reference in its entirety). In the majority of COVID-19 patients, immune mechanisms result in clearance of SARS-CoV-2 and resolution of inflammatory responses. Inflammatory cytokines, such as type I interferon, can reduce coronavirus loads during early pulmonary infection but can lead to increased lung pathology during later stages (Ref. 3; incorporated by reference in its entirety). Dysfunctional immune responses, sometimes referred to as cytokine storm, can lead to progressive lung injury and death (Ref. 4; incorporated by reference in its entirety). While the magnitude of early and late inflammatory responses to SARS-CoV-2 infection contribute to the wide range of clinical outcomes in COVID-19 patients, the underlying reasons for these disparities remain largely unexplained (Ref. 5; incorporated by reference in its entirety).

### SUMMARY

Provided herein are biomarkers associated with mortality in COVID-19 patients. In particular, provided herein are fecal biomarkers that correlate with elevated risk of mortality in subjects suffering from COVID-19 with severe respiratory insufficiency, and methods of treating COVID-19 based thereon.

In some embodiments, provided herein are methods of assessing the risk of mortality for a subject comprising assessing the level of one or more biomarkers of mortality in the gut of the subject. In some embodiments, the subject suffers from respiratory insufficiency. In some embodiments, the subject suffers from severe respiratory insufficiency. In some embodiments, the subject suffers from COVID-19. In some embodiments, the subject suffers from COVID-19 acute respiratory distress syndrome (ARDS). In some embodiments, the subject is hospitalized. In some embodiments, the subject is ventilated. In some embodiments, the level of the one or more biomarkers of mortality are assessed in a fecal sample from the subject. In some embodiments, the one or more biomarkers of mortality are selected from microbiomic and metabolomic biomarkers.

In some embodiments, one or more microbiomic biomarkers, selected from the level of one or more bacterial species belonging to the families Bacteroidaceae, Lachnospiraceae and Enterobacteriaceae or the phylum Proteobacteria, are assessed. In some embodiments, an increased level of the one or more bacterial species belonging to the families Bacteroidaceae and Lachnospiraceae is associated with survival and a decreased level of the one or more bacterial species belonging to the families Bacteroidaceae and Lachnospiraceae is associated with mortality. In some embodiments, a decreased level of the one or more bacterial species belonging to the family Enterobacteriaceae or phylum Proteobacteria is associated with survival and an increased level of the one or more bacterial species belonging to the family Enterobacteriaceae or phylum Proteobacteria is associated with mortality.

In some embodiments, the level of one or more metabolomic biomarkers is assessed by mass spectrometric analysis of a sample from the subject. In some embodiments, one or more metabolomic biomarkers, selected from the level of one or more of indole-3-carboxaldehyde, deaminotyrosine, deoxucholic acid, 3-oxodeoxycholic acid, 3-oxochenodeoxycholic acid, lithocholic acid, 3-aminoisobutyrate, serine, isodeoxycholic acid, toluate, and allolithocholic acid, are assessed. In some embodiments, an increased level of one or more of indole-3-carboxaldehyde, deaminotyrosine, deoxucholic acid, 3-oxodeoxycholic acid, 3-oxochenodeoxycholic acid, lithocholic acid, 3-aminoisobutyrate, serine, and isodeoxycholic acid is associated with survival and a decreased level of one or more of indole-3-carboxaldehyde, deaminotyrosine, deoxucholic acid, 3-oxodeoxycholic acid, 3-oxochenodeoxycholic acid, lithocholic acid, 3-aminoisobutyrate, serine, and isodeoxycholic acid is associated with mortality. In some embodiments, the increased level is above a threshold level and the decreased level is below the threshold level. In some embodiments, a decreased level of one or both of toluate and allolithocholic acid is associated with survival and an increased level of one or both of toluate and allolithocholic acid is associated with mortality. In some embodiments, the increased level is above a threshold level and the decreased level is below the threshold level. In some embodiments, the level of two or more of deoxycholic acid, isodeoxycholic acid, lithocholic acid, and desaminotyrosine are assessed, wherein high levels of the two or more of deoxycholic acid, isodeoxycholic acid, lithocholic acid, and desaminotyrosin is associated with a high risk of mortality. In some embodiments, high levels are levels above a threshold value. In some embodiments, the threshold is between 70 μM and 110 μM for deoxycholic acid, between 0.8 M and 1.2 μM for isodeoxycholic acid, between 220 μM and 300 μM for lithocholic acid, and between 10 μM and 30 μM for desaminotyrosin. In some embodiments, the threshold is about 90 μM for deoxycholic acid, about 1 μM for isodeoxycholic acid, about 258 μM for lithocholic acid, and about 21 μM for desaminotyrosin. In some embodiments, methods comprise assessing the levels of each of deoxycholic acid, isodeoxycholic acid, lithocholic acid, and desaminotyrosine.

In some embodiments, provided herein is a method of treating a subject suffering from COVID-19 acute respiratory distress syndrome (ARDS), comprising assessing the subject's risk of mortality by the methods of one of claims 1-23, wherein if the subject is determined to be at increased risk of mortality fecal augmentation therapy is administered, and wherein if the subject is determined to be at decreased risk of mortality fecal augmentation therapy is not administered.

### BRIEF DESCRIPTION OF THE DRAWINGS

FIG. 1A-E. Fecal microbiome composition in patients with severe COVID-19 stratified by mortality. FIG. 1A) Shotgun metagenomics-based taxonomy plots stratified by survival. FIG. 1B) Alpha diversity (Inverse Simpson) plot stratified by survival where colored bars represent the average Inverse Simpson value while gray boxes denote 95% confidence intervals. FIG. 1C) Uniform Manifold Approximation and Projection (UMAP) from shotgun metagenomics-based taxonomy, colored by survival with centroids ellipses. FIG. 1D) UMAP colored by expansion of Enterococcus (green), Proteobacteria (red), both Enterococcus and Proteobacteria (red and green halves), and no expansions (gray) with centroids and ellipse. FIG. 1E) A Linear discriminant analysis effect size (LEfSe) showing the significant (p≤0.05) effect sizes of taxa between survival groups.

FIG. 2A-D. Representation of genes with antibiotic resistance, toxins and metabolite production stratified by Mortality. FIG. 2A displays genes encoding for antibiotic resistance. FIG. 2B displays genes encoding for bacteriocins. FIG. 2C displays genes encoding for toxins/hemolysins/cytolysins. FIG. 2D displays the genes responsible for bile acid conversion, as well as butyrate-related enzymes and desaminotyrosine. Genes in FIGS. 2A, 2B, and 2D were quantified using RPKM values while toxin genes in FIG. 2C were determined as presence/absence. P-values were obtained from Wilcoxon rank-sum test (FIGS. 2A, 2B, and 2D) and chi-squared test (FIG. 2C).

FIG. 3A-B. Qualitative and quantitative fecal metabolomic analyses. FIG. 3A) Volcano plot of normalized metabolite concentrations, where values above the horizontal line (unadjusted p-value >0.05) and log 2 fold-change values >=1 were used to identify metabolites associated with survival. FIG. 3B) Metabolites identified in panel A were quantified in fecal extracts by LC-MS and are shown as boxplots with Wilcoxon rank-sum tests and their associated p-values. Bile acids (light gray strips) and desaminotyrosine and indole-3-carboxaldehyde (dark gray strips) are in units of M.

FIG. 4A-B. A Microbiome Metabolite Profile (MMP) predicts mortality in patients with severe COVID-19. FIG. 4A) Area under the curve (AUC) for the microbiome metabolite profile and mortality. AUC=0.744. Positive predictive value (PPV)=0.75 and negative predictive value (NPV=0.67). FIG. 4B) Kaplan-Meier survival curves stratified by low MMP scores (0-1) versus high MMP score (2-4) are plotted. Time in days is presented on the X-Axis. Log-rank test was used to assess significant differences.

FIG. 5. Progression of Respiratory Failure Stratified by Trajectory. Each row represents an individual patient course. Blue dots represent initial fecal samples collected within 3 days of ICU admission. Figure is stratified by patients who transitioned from high flow nasal cannula to low flow nasal cannula versus those who progressed to endotracheal intubation and received mechanical ventilation. Patients in whom a transitioned could not be identified were labeled unclassifiable. HFNC=High Flow Nasal Cannula, ETT=Endotracheal Tube, LTACH=Long Term Acute Care Hospital, SNF=Skilled Nursing Facility, NIPPV=Non-invasive Positive Pressure Ventilation.

FIG. 6. Consort diagram for study enrollment.

FIG. 7. KEGG pathway analysis stratified by survival outcome. KEGG pathways (Rank 2) were calculated as a total percent of all pathways encoded and plotted as values per each rank 2 category. There were no statistical differences across all KEGG pathways shown.

FIG. 8. Heatmap of ninety-two relatively quantified fecal metabolites. Dark cyan colors correlate to low log 2 fold-change values relative to the median value per compound, while dark red colors correlate to high log 2 fold-change values relative to the median value per compound. Missing values are shown in gray.

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise. Thus, for example, reference to “a metabolite” is a reference to one or more metabolites and equivalents thereof known to those skilled in the art, and so forth.

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

The term “about” allows for a degree of variability in a value or range. As used herein, the term “about: refers to values within 10% of the recited value or range (e.g., about 50 is the equivalent of 45-55). As used herein, the term “microbiome” refers to the microbes (e.g., bacteria, fungi, protists) their genetic elements (genomes) in a defined environment. The term “gut microbiome” refers to the microbes present in the digestive tract of a subject. A “microbimic analysis” refers to the use of any suitable techniques to identify the presence/absence/level of one or more (e.g., all) microbes within a sample or environment. The term “microbiomic biomarker” refers to a species or group of microbes, the presence/absence/level of which in a sample from a subject is indicative/prognostic/diagnostic of a condition/outcome in the subject.

As used herein, the term “metabolome” refers to the complete set of small-molecule metabolites (e.g., metabolic intermediates, hormones signaling molecules, secondary metabolites, etc.) found within a defined biological environment or sample. The term “gut metabolome” refers to the metabolites present in the digestive tract of a subject. A “metabolomic analysis” refers to the use of any suitable techniques to identify the presence/absence/level of one or more (e.g., all) of the metabolites within a sample or environment. The term “metabolomic biomarker” refers to a small molecule metabolite, the presence/absence/level of which in a sample from a subject is indicative/prognostic/diagnostic of a condition/outcome in the subject. As used herein, the term “subject” broadly refers to any animal, including but not limited to, human and non-human animals (e.g., dogs, cats, cows, horses, sheep, poultry, fish, crustaceans, etc.). As used herein, the term “patient” typically refers to a subject that is being treated for a disease or condition.

As used herein, the terms “administration” and “administering” refer to the act of giving a drug, prodrug, or other agent, or therapeutic treatment to a subject or in vivo, in vitro, or ex vivo cells, tissues, and organs. Exemplary routes of administration to the human body can be by parenteral administration (e.g., intravenously, subcutaneously, etc.), oral administration, rectal administration, etc..

As used herein, the term “effective amount” refers to the amount of a composition sufficient to effect beneficial or desired results. An effective amount can be administered in one or more administrations, applications or dosages and is not intended to be limited to a particular formulation or administration route.

As used herein, the terms “co-administration” and “co-administering” refer to the administration of at least two agent(s) or therapies to a subject. In some embodiments, the co-administration of two or more agents or therapies is concurrent (e.g., in a single formulation/composition or in separate formulations/compositions). In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s), and/or when co-administration of two or more agents results in sensitization of a subject to beneficial effects of one of the agents via co-administration of the other agent.

As used herein, the term “pharmaceutical composition” refers to the combination of an active agent with a carrier, inert or active, making the composition especially suitable for diagnostic or therapeutic use in vitro, in vivo or ex vivo.

As used herein, the term “prognosis” refers to risk prediction of the severity of disease or of the probable course and clinical outcome associated with a disease. Thus, the term “method of prognosis” as used herein refers to methods by which the skilled person can estimate and/or determine a probability that a given outcome will occur. The outcome to which the prognosis relates may be morbidity and/or mortality.

The terms “stratification” or “stratifying” as used herein refer to the division of a population into subpopulations on the basis of specified criteria. More particularly, it refers to the division of a cohort of subjects into at least two groups on the basis of specific criteria, which in the context of the present invention comprise or consist of the results of the method of analysis. Optionally, subjects may be stratified based on the likelihood of survival/mortality.

### DETAILED DESCRIPTION

Provided herein are biomarkers associated with mortality in COVID-19 patients. In particular, provided herein are fecal biomarkers that correlate with elevated risk of mortality in subjects suffering from COVID-19 with severe respiratory insufficiency, and methods of treating COVID-19 based thereon.

Studies in mice have demonstrated that the intestinal microbiome impacts pulmonary immune defenses against respiratory viral infections (Refs. 8, 9; incorporated by reference in their entireties). Commensal bacterial species produce metabolites that can activate systemic immune defenses and modulate inflammatory responses (Refs. 10-11; incorporated by reference in their entireties) and a diverse microbiome and its metabolic products can stimulate the host immune system and support immune homeostasis (Ref. 12; incorporated by reference in its entirety). Production of bacterial metabolites, such as butyrate and secondary bile acids, can modulate development of inflammatory and regulatory T cell populations and provide defense against pathogens (Refs. 13-17; incorporated by reference in their entireties). Microbiota-derived metabolites are increasingly recognized as contributing to respiratory antiviral defense (Ref. 11; incorporated by reference in its entirety). For example, desaminotyrosine, a metabolite produced by a subset of intestinal microbes, has been shown in mice to enhance antiviral type I IFN responses and pulmonary clearance of influenza virus (Ref. 18; incorporated by reference in its entirety). In patients undergoing hematopoietic cell transplantation (HCT), microbiota analyses revealed a five-fold increase in progression of viral respiratory tract infections among patients with reduced abundance of butyrate-producing commensal bacterial species (Ref. 19; incorporated by reference in its entirety). The association between butyrate-producing bacteria and progression of respiratory viral illness was also seen in patients following renal transplantation (Ref. 20; incorporated by reference in its entirety).

Metagenomic sequencing studies have demonstrated that fecal microbiome compositions of COVID-19 patients are distinct from healthy subjects (Ref. 21; incorporated by reference in its entirety) and have revealed differences in microbiome composition and circulating markers of inflammation in patients with mild, moderate or severe respiratory disease (Refs. 22-25; incorporated by reference in their entireties). Among patients with severe COVID-19, however, it remains unclear whether microbiome compositions and microbially-derived, immunomodulatory metabolites are associated with progression of COVID-19-associated respiratory failure and mortality. To address this, experiments were conducted during development of embodiments herein to profile fecal microbiomes and metabolomes of patients admitted to the intensive care unit with COVID-19 and to correlate these profiles with improvement or worsening of respiratory function and mortality.

The high mortality associated with COVID-19 results from viral injury to lung tissue, overly robust inflammatory responses, and subsequent alveolar damage. While the relative contributions of these factors vary from one patient to another, autopsy studies indicate that in fatal COVID-19, all three play a role (Refs. 2-3; incorporated by reference in their entireties). A multifaceted approach was used to assess the integrity of the fecal microbiome during COVID-19 associated critical illness and found that the intestinal microbiome and its metabolites at the time of ICU admission are independently associated with the need for intubation and survival. Microbiota-derived metabolites contribute to viral clearance, modulation of inflammation and reestablishment of epithelial integrity. While conventional assessments of microbial α-diversity did not distinguish survivors from patients who died, in multivariable models, a subset of microbially derived fecal metabolites correlated with survival. Furthermore, the associations found in this study persisted after taking antibiotic treatment, duration of symptoms, and severity of illness into account.

While previous studies have correlated increased frequencies of Proteobacteria and reduced frequencies of obligate anaerobic commensal species with SARS-CoV-2 infection (Refs. 5, 22, 24; incorporated by reference in their entireties) the experiments conducted during development of embodiments herein provide the first correlation with COVID-19 mortality and need for intubation. Experimental SARS-CoV-2 infection of rhesus macaques resulted in expansion of Proteobacteria during peak infection (Ref. 26; incorporated by reference in its entirety) indicating that the microbiota compositional changes detected in these patients result from the systemic viral infection.

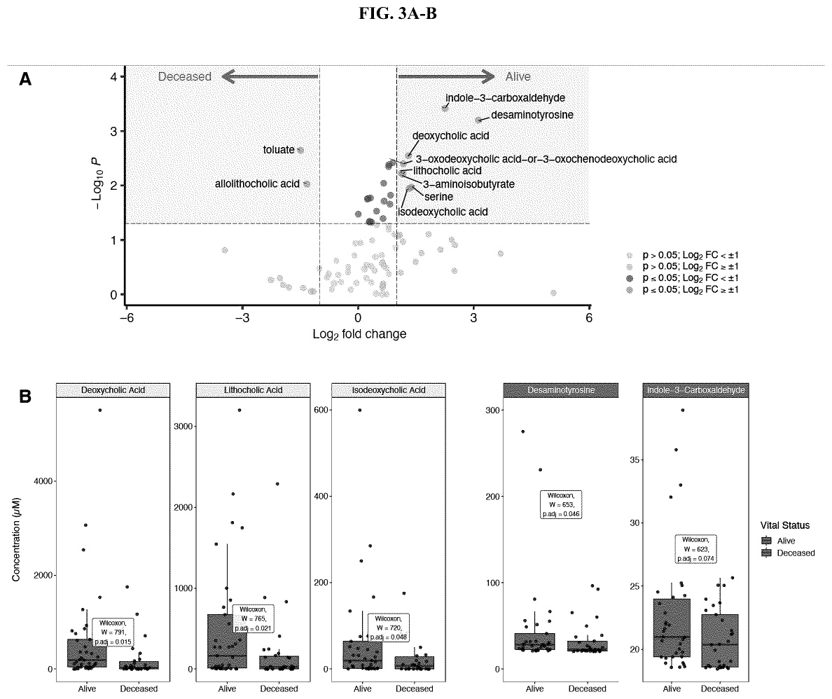

Immune responses during early stages of viral infection and regulation of lung inflammation at later stages are likely impacted by the microbiome and its metabolites. The finding that increased concentrations of fecal secondary bile acids are associated with improved outcomes may result from their impact on differentiation of CD4 Th17 and Treg cells (Refs. 16, 17, 27; incorporated by reference in their entireties). The secondary bile acid deoxycholate is produced by a subset of commensal bacteria (Ref. 28; incorporated by reference in its entirety) and is further converted by bacterial species expressing 3αHSDH and 3βHSDH to isodeoxycholate, which is less toxic to mammalian cells and commensal bacterial species (Ref. 29; incorporated by reference in its entirety). Isodeoxycholate also renders dendritic cells less immunostimulatory, thereby enhancing generation of peripherally induced T regulatory cells (Refs. 11, 16; incorporated by reference in their entireties). Some bacterial strains belonging to the Bacteroidales order express 5AR and 3β-HSDH, enabling them to generate alloisolithocholate from bile acid intermediates along the Bai pathway (Ref. 30; incorporated by reference in its entirety). Importantly, alloisolithocholate can inhibit gram positive pathogens and also enhance the development of T regulatory cells (Refs. 17, 27, 30; incorporated by reference in their entireties). The finding that the frequency of genes encoding enzymes that mediate secondary bile acid synthesis did not differ between patients with respect to mortality while fecal secondary bile acid concentrations did (FIG. 3), indicates that metabolic profiling represents a more sensitive measure of the potential impact of the microbiome on host physiology and immune defense than metagenomic sequencing. It also demonstrates that metabolite production is not only dependent on the presence of genes but host factors such as diet, inflammation, and the presence of essential co-factors. Additionally, a functional assessment of the integrity of the microbiome through metabolites is advantageous clinically as they can be rapidly quantified.

Experiments conducted during development of embodiments herein demonstrate that microbiome composition and a subset of microbiota-derived metabolites are independently associated, for example, with survival and the trajectory of respiratory failure among patients admitted to the ICU with COVID-19.

The present disclosure provides a group of prognostic biomarkers (e.g., microbiomic biomarkers, metabolomic biomarkers, etc.) usable for assessing the relative risk of mortality or survival of a subject suffering from COVID-19 and/or respiratory failure. A method for assessing the risk of mortality for a subject suffering from COVID-19 and/or respiratory failure the group of prognostic biomarkers (e.g., microbiomic biomarkers, metabolomic biomarkers, etc.) is also provided. For example, the method provided by the present disclosure is a non-invasive approach that utilizes body fluid or other sample (e.g., fecal samples) from a patient (e.g., hospitalized, in intensive care, ventilated, etc.) suffering from COVID-19 and/or respiratory failure to determine the risk of mortality for the patient.

Experiments conducted during development of embodiments herein identified prognostic biomarkers (e.g., microbiomic biomarkers, metabolomic biomarkers, etc.) that are present in different levels in subjects likely to survive COVID-19 (e.g., severe COVID-19) and/or respiratory failure and subject likely not to survive COVID-19 (e.g., severe COVID-19) and/or respiratory failure. In some embodiments, analysis of such biomarkers in a sample (e.g., fecal sample) from a subject provides a prognosis for the likelihood of survival/mortality and indicates treatment courses of action that should be taken on behalf of the subject. In some embodiments, a panel of biomarkers is provided, wherein each individual biomarker is indicative of the survival/mortality of the subject, but together provide an enhanced determination as to the risk of mortality for the subject. In some embodiments, methods are provided for assessing the levels of the various biomarkers herein (e.g., microbiomic biomarkers, metabolomic biomarkers, etc.). In some embodiments, the levels of one or more biomarkers herein are compared to controls and/or threshold values. In some embodiments, methods are provided for analyzing the levels of multiple biomarkers and providing a prognosis (e.g., low likelihood of mortality, intermediate likelihood of mortality, high likelihood of mortality) and/or a treatment course of action based thereupon. In some embodiments, a score is provided based on the levels of one or more biomarkers. In some embodiments, the score indicates the risk of mortality.

In some embodiments, provided herein are species or taxonomic groups of microbes (e.g., bacteria), the presence/absence or level of which is a subject or sample from the subject is prognostic of the relative likelihood of survival/mortality of a subject suffering from COVID-19 and/or respiratory failure. In some embodiments, provided herein are panels of such microbiotic biomarkers.

Experiments conducted during development of embodiments herein demonstrate that the levels of bacterial species belonging to the families Bacteroidaceae, Lachnospiraceae and Enterobacteriaceae or the phylum Proteobacteria in a stool sample from a subject suffering from COVID-19 (e.g., severe COVID-19) and/or severe respiratory insufficiency (e.g., severe respiratory insufficiency) are prognostic for the likelihood of survival/mortality of the subject.

In some embodiments, an increased level (e.g., above a threshold abundance) of one or more bacterial species belonging to the families Bacteroidaceae and Lachnospiraceae is associated with survival for a subject suffering from COVID-19 (e.g., severe COVID-19) and/or severe respiratory insufficiency (e.g., severe respiratory insufficiency). In some embodiments, a decreased level (e.g., below a threshold abundance) of the one or more bacterial species belonging to the families Bacteroidaceae and Lachnospiraceae is associated with mortality.

In some embodiments, a decreased level (e.g., below a threshold abundance) of one or more bacterial species belonging to the family Enterobacteriaceae or phylum Proteobacteria is associated with survival for a subject suffering from COVID-19 (e.g., severe COVID-19) and/or severe respiratory insufficiency (e.g., severe respiratory insufficiency). In some embodiments, an increased level (e.g., above a threshold abundance) of the one or more bacterial species belonging to the family Enterobacteriaceae or phylum Proteobacteria is associated with mortality.

In some embodiments, a microbiomic biomarker herein (e.g., on a panel of biomarkers, detected or quantified in methods herein, etc.) is a bacteria from the family Bacteroidaceae. In some embodiments, the Bacteroidaceae bacteria are of one or more genera selected from Bacteroides, Acetofilamentum, Acetomicrobium, Acetothermus, and/or Anaerorhabdus. Examples of bacteria from the family Bacteroidaceae that may be present on biomarker panels herein and/or may be detected/quantitated in embodiments herein include, but are not limited to: A. rigidum, A. paucivorans, B. acidifaciens, B. barnesiaes, B. caccae, B. caecicola, B. caecigallinarum, B. cellulosilyticus, B. cellulosolvens, B. clarus, B. coagulans, B. coprocola, B. coprophilus, B. coprosuis, B. dorei, B. eggerthii, B. gracilis, B. faecichinchillae, B. faecis, B. finegoldii, B. fluxus, B. fragilis, B. galacturonicus, B. gallinaceum, B. gallinarum, B. goldsteinii, B. graminisolvens, B. helcogene, B. intestinalis, B. luti, B. massiliensis, B. nordii, B. oleiciplenus, B. oris, B. ovatus, B. paurosaccharolyticus, B. plebeius, B. polypragmatus, B. propionicifaciens, B. putredinis, B. pyogenes, B. reticulotermitis, B. rodentium, B. salanitronis, B. salyersiae, B. sartorii, B. sediment, B. stercoris, B. suis, B. tectus, B. thetaiotaomicron, B. uniformis, B. vulgatus, and B. xylanisolvens.

In some embodiments, a microbiomic biomarker herein (e.g., on a panel of biomarkers, detected or quantified in methods herein, etc.) is a bacteria from the family Lachnospiraceae. In some embodiments, the Lachnospiraceae bacteria are of one or more genera selected from Abyssivirga, Acetatifactor, Acetitomaculum, Agathobacter, Anaerostipes, Blautia, Butyrivibrio, Catonella, Cellulosilyticum, Coprococcus, Dorea, Hespellia Johnsonella, Lachnoanaerobaculum, Lachnobacterium, Lachnospira, Marvinbryantia, Mobilitalea, Moryella, Oribacterium, Parasporobacterium, Pseudobutyrivibrio, Robinsoniella, Roseburia, L- Ruminococcus, Shuttleworthia, Sporobacterium, Stomatobaculum, and/or Syntrophococcus.

In some embodiments, a microbiomic biomarker herein (e.g., on a panel of biomarkers, detected or quantified in methods herein, etc.) is a bacteria from the family Enterobacteriaceae. In some embodiments, the Enterobacteriaceae bacteria are of one or more genera selected from Biostraticola, Buttiauxella, Cedecea, Citrobacter, Cronobacter, Enterobacillus, Enterobacter, Escherichia, Franconibacter, Gibbsiella, Izhakiella, Klebsiella, Kluyvera, Kosakonia, Leclercia, Lelliottia, Limnobaculum, Mangrovibacter, Metakosakonia, Phytobacter, Pluralibacter, Pseudescherichia, Pseudocitrobacter, Raoultella, Rosenbergiella, Saccharobacter, Salmonella, Scandinavium, Shigella, Shimwellia, Siccibacter, Trabulsiella, and/or Yokenella.

In some embodiments, a microbiomic biomarker herein (e.g., on a panel of biomarkers, detected or quantified in methods herein, etc.) is a bacteria from the phylum Proteobacteria. In some embodiments, the Proteobacteria bacteria are of one or more classes selected from Acidithiobacillia, Alphaproteobacteria, Betaproteobacteria, Gammaproteobacteria, Hydrogenophilalia, and/or Zetaproteobacteria.

In some embodiments, microbiomic biomarkers are assessed (e.g., quantified) is a sample form a subject by any suitable method. In some embodiments, a sample is tested to determine the microbiotic makeup of the sample. In some embodiments, specific species or taxonomic groups of bacteria are tested for (e.g., species of taxa that are prognostic of survival/mortality risk in subjects suffering from COVID-19 (e.g., severe COVID-19) and/or severe respiratory insufficiency (e.g., severe respiratory insufficiency)). In some embodiments, intact bacteria are detected (e.g., by detecting surface polypeptides or markers). In other embodiments, bacteria are lysed and nucleic acids or proteins (e.g., corresponding to genes specific to the species of bacteria) are detected. In some embodiments, bacteria are identified using detection reagents (e.g., a probe, a microarray, e.g., an amplification primer) that specifically interact with a nucleic acid that identifies a particular species or taxa of bacteria.

Some embodiments comprise use of nucleic acid sequencing to detect, quantify, and/or identify gut microbiota. The term “sequencing,” as used herein, refers to a method by which the identity of at least 10 consecutive nucleotides (e.g., the identity of at least 20, at least 50, at least 100, or at least 200 or more consecutive nucleotides) of a polynucleotide are obtained. The term “next-generation sequencing” refers to the so-called parallelized sequencing-by-synthesis, sequencing-by-ligation platforms, nanopore sequencing methods, or electronic-detection based methods that will be understood in the field.

Some embodiments comprise acquiring a gut microbiota sample from a subject. As used herein, “gut microbiota sample” refers to a biological sample comprising a plurality of heterogeneous nucleic acids produced by a subject's gut microbiota. Fecal samples are commonly used in the art to sample gut microbiota. Methods for obtaining a fecal sample from a subject are known in the art and include, but are not limited to, rectal swab and stool collection. Suitable fecal samples may be freshly obtained or may have been stored under appropriate temperatures and conditions known in the art. Methods for extracting nucleic acids from a fecal sample are also well known in the art. The extracted nucleic acids may or may not be amplified prior to being used as an input for profiling the relative abundances of bacterial taxa, depending upon the type and sensitivity of the downstream method. When amplification is desired, nucleic acids may be amplified via polymerase chain reaction (PCR). Methods for performing PCR are well known in the art. Selection of nucleic acids or regions of nucleic acids to amplify are discussed above. The nucleic acids comprising the nucleic acid sample may also be fluorescently or chemically labeled, fragmented, or otherwise modified prior to sequencing or hybridization to an array as is routinely performed in the art.

In some embodiments, nucleic acids are amplified using primers that are compatible with use in, e.g., Illumina's reversible terminator method, Roche's pyrosequencing method (454), Life Technologies's sequencing by ligation (the SOLiD platform) or Life Technologies's Ion Torrent platform. Examples of such methods are described in the following references: Margulies et al (Nature 2005 437: 376-80); Ronaghi et al (Analytical Biochemistry 1996 242: 84-9); Shendure et al (Science 2005 309: 1728-32); Imelfort et al (Brief Bioinform. 2009 10:609-18); Fox et al (Methods Mol Biol. 2009; 553:79-108); Appleby et al (Methods Mol Biol. 2009; 513: 19-39) and Morozova et al (Genomics. 2008 92:255-64), which are incorporated by reference for the general descriptions of the methods and the particular steps of the methods, including all starting products, reagents, and final products for each of the steps.

In another embodiment, the isolated microbial DNA may be sequenced using nanopore sequencing (e.g., as described in Soni et al. Clin Chem 2007 53: 1996-2001, or as described by Oxford Nanopore Technologies). Nanopore sequencing technology is disclosed in U.S. Pat. Nos. 5,795,782, 6,015,714, 6,627,067, 7,238,485 and 7,258,838 and U.S. Pat Appln Nos. 2006003171 and 20090029477.

The isolated microbial fragments may be sequenced directly or, in some embodiments, the isolated microbial fragments may be amplified (e.g., by PCR) to produce amplification products that sequenced. In certain embodiments, amplification products may contain sequences that are compatible with use in, e.g., Illumina's reversible terminator method, Roche's pyrosequencing method (454), Life Technologies' sequencing by ligation (the SOLiD platform) or Life Technologies' Ion Torrent platform, as described above. Embodiments herein are not limited by the techniques used to identify and/or quantify the bacteria present in a sample.

In some embodiments, provided herein are species or taxonomic groups of microbes (e.g., bacteria), the presence/absence or level of which is a subject or sample from the subject is prognostic of the relative likelihood of survival/mortality of a subject suffering from COVID-19 and/or respiratory failure. In some embodiments, provided herein are panels of such microbiotic biomarkers.

Experiments conducted during development of embodiments herein demonstrate that the levels of various small molecule metabolites present in biological samples (e.g., stool sample, rectal swab, etc.) from a subject suffering from COVID-19 (e.g., severe COVID-19) and/or severe respiratory insufficiency (e.g., severe respiratory insufficiency) correlate with and are prognostic of the likelihood of survival/mortality of the subject. In some embodiments, provided herein are panels of metabolites, the levels of which in biological samples (e.g., stool sample, rectal swab, etc.) from a subject suffering from COVID-19 (e.g., severe COVID-19) and/or severe respiratory insufficiency (e.g., severe respiratory insufficiency) correlate with the rate of survival/mortality of the subject. In some embodiments, methods are provided of assessing the levels of such metabolomic biomarkers in a sample biological samples (e.g., stool sample, rectal swab, etc.) from a subject suffering from COVID-19 (e.g., severe COVID-19) and/or severe respiratory insufficiency (e.g., severe respiratory insufficiency). In some embodiments, the metabolomic biomarkers in the panels herein and/or assessed herein include one or more biomarkers selected from indole-3-carboxaldehyde, deaminotyrosine, deoxucholic acid, 3-oxodeoxycholic acid, 3-oxochenodeoxycholic acid, lithocholic acid, 3-aminoisobutyrate, serine, isodeoxycholic acid, toluate, and allolithocholic acid.

In some embodiments, the levels of one or more of indole-3-carboxaldehyde, deaminotyrosine, deoxucholic acid, 3-oxodeoxycholic acid, 3-oxochenodeoxycholic acid, lithocholic acid, 3-aminoisobutyrate, serine, isodeoxycholic acid, toluate, and allolithocholic acid in a sample are quantitated. In some embodiments, an increased level (e.g., relative to a control or a threshold level) of one or more of indole-3-carboxaldehyde, deaminotyrosine, deoxucholic acid, 3-oxodeoxycholic acid, 3-oxochenodeoxycholic acid, lithocholic acid, 3-aminoisobutyrate, serine, and isodeoxycholic acid in a biological sample (e.g., stool sample, rectal swab, etc.) from a subject suffering from COVID-19 (e.g., severe COVID-19) and/or severe respiratory insufficiency (e.g., severe respiratory insufficiency) is correlated with survival. In some embodiments, a decreased level (e.g., relative to a control or a threshold level) of one or more of indole-3-carboxaldehyde, deaminotyrosine, deoxucholic acid, 3-oxodeoxycholic acid, 3-oxochenodeoxycholic acid, lithocholic acid, 3-aminoisobutyrate, serine, and isodeoxycholic acid in a biological sample (e.g., stool sample, rectal swab, etc.) from a subject suffering from COVID-19 (e.g., severe COVID-19) and/or severe respiratory insufficiency (e.g., severe respiratory insufficiency) is correlated with mortality. In some embodiments, a decreased level (e.g., relative to a control or a threshold level) of one or both of toluate and allolithocholic acid in a biological sample (e.g., stool sample, rectal swab, etc.) from a subject suffering from COVID-19 (e.g., severe COVID-19) and/or severe respiratory insufficiency (e.g., severe respiratory insufficiency) is correlated with survival. In some embodiments, an increased level (e.g., relative to a control or a threshold level) of one or both of toluate and allolithocholic acid in a biological sample (e.g., stool sample, rectal swab, etc.) from a subject suffering from COVID-19 (e.g., severe COVID-19) and/or severe respiratory insufficiency (e.g., severe respiratory insufficiency) is correlated with mortality. In some embodiments, an increased level is any amount above a threshold level and a decreased level is ay amount below the threshold level.

In some embodiments, a method comprises detecting a level of one or more (e.g., 1, 2, 3, 4, or ranges therebetween) of deoxycholic acid, isodeoxycholic acid, lithocholic acid, and desaminotyrosine in a biological sample from a subject, wherein high levels of one or more (e.g., 1, 2, 3, 4, or ranges therebetween) of deoxycholic acid, isodeoxycholic acid, lithocholic acid, and/or desaminotyrosin is associated with a high risk of mortality. In some embodiments, high levels are defined as levels above a threshold value. In some embodiments, the threshold is (about) 50 μM, 60 μM, 70 μM, 80 μM, 90 μM, 100 μM, 110 μM, 120 μM, 130 μM, 140 μM or 150 μM (or any values or ranges therebetween) for deoxycholic acid. In some embodiments, the threshold is (about) 0.5 μM, 0.6 μM, 0.7 μM, 0.8 μM, 0.9 μM, 1.0 μM, 1.1 μM, 1.2 μM, 1.3 μM, 1.4 μM, 1.5 μM (or any values or ranges therebetween) for isodeoxycholic acid. In some embodiments, the threshold is (about) 200 μM, 210 μM, 220 μM, 230 μM, 240 μM, 250 μM, 260 μM, 270 μM, 280 μM, 290 μM, 300 μM, 310 μM, 320 μM, or 330 μM (or any values or ranges therebetween) for lithocholic acid. In some embodiments, the threshold is (about) 5 μM, M, 15 μM, 20 μM, 25 μM, 30 μM, 35 μM, or 40 μM (or any values or ranges therebetween) for desaminotyrosin. In some embodiments, the threshold is about 90 μM for deoxycholic acid, about 1 μM for isodeoxycholic acid, about 258 μM for lithocholic acid, and about 21 μM for desaminotyrosin.

In some embodiments, a metabolomics screen is performed on a sample from a subject identify, quantify, etc. various metabolites present. In some embodiments, a metabolomic screen is performed to detect and/or quantify as many metabolites as are detectable by the methods used. In some embodiments, one or more key metabolites (e.g., metabolites identified herein) are detected and/or quantified. In some embodiments, 2 or more metabolites are detected an/or quantified (e.g., 2, 3, 4, 5, 6, 7, 8, 9, 10, 12, 15, 20, 25, 30, 35, 40, 45, 50, 75, 100, or more).

In some embodiments, any technique and/or instrumentation suitable for detecting/quantifying small molecules in a complex environment may find use in embodiments herein. In some embodiments, analytical platforms (e.g.,. High-throughput platforms, automated platforms, etc.) utilizing nuclear magnetic resonance (NMR) spectroscopy, gas chromatography (GC), liquid chromatography (LC), and/or mass spectrometry (MS) are employed to measure the metabolites within a biological sample. In some embodiments, NMR, GC, and/or LC coupled to MS is utilized.

Mass spectrometry can accurately identify/quantify thousands of metabolites within complex biological samples. In some embodiments, metabolites are detected/quantified in a biological sample using MS techniques, such as MALDI/TOF (time-of-flight), SELDI/TOF, liquid chromatography-mass spectrometry (LC-MS), gas chromatography-mass spectrometry (GC-MS), high performance liquid chromatography-mass spectrometry (HPLC-MS), capillary electrophoresis-mass spectrometry, nuclear magnetic resonance spectrometry, tandem mass spectrometry (e.g., MS/MS, MS/MS/MS, ESI-MS/MS etc.), secondary ion mass spectrometry (SIMS), or ion mobility spectrometry (e.g. GC-IMS, IMS-MS, LC-IMS, LC-IMS-MS etc.). Mass spectrometry methods are well known in the art and have been used to quantify and/or identify biomolecules, such metabolites.

In certain embodiments, a gas phase ion spectrophotometer is used. In other embodiments, laser-desorption/ionization mass spectrometry is used to identify metabolites. Modern laser desorption/ionization mass spectrometry (“LDI-MS”) can be practiced in two main variations: matrix assisted laser desorption/ionization (“MALDI”) mass spectrometry and surface-enhanced laser desorption/ionization (“SELDI”). In MALDI, the metabolite is mixed with a solution containing a matrix, and a drop of the liquid is placed on the surface of a substrate. The matrix solution then co-crystallizes with the biomarkers. The substrate is inserted into the mass spectrometer. Laser energy is directed to the substrate surface where it desorbs and ionizes the proteins without significantly fragmenting them. However, MALDI has limitations as an analytical tool. It does not provide means for fractionating the biological fluid, and the matrix material can interfere with detection, especially for low molecular weight analytes. In SELDI, the substrate surface is modified so that it is an active participant in the desorption process. In one variant, the surface is derivatized with adsorbent and/or capture reagents that selectively bind the biomarker of interest. In another variant, the surface is derivatized with energy absorbing molecules that are not desorbed when struck with the laser. In another variant, the surface is derivatized with molecules that bind the biomarker of interest and that contain a photolytic bond that is broken upon application of the laser. In each of these methods, the derivatizing agent generally is localized to a specific location on the substrate surface where the sample is applied. The two methods can be combined by, for example, using a SELDI affinity surface to capture an analyte (e.g. biomarker) and adding matrix-containing liquid to the captured analyte to provide the energy absorbing material.

For additional information regarding mass spectrometers, see, e.g., Principles of Instrumental Analysis, 3rd edition., Skoog, Saunders College Publishing, Philadelphia, 1985; and Kirk-Othmer Encyclopedia of Chemical Technology, 4.sup.th ed. Vol. 15 (John Wiley & Sons, New York 1995), pp. 1071-1094; incorporated by reference in their entireties.

In some embodiments, the data from mass spectrometry is represented as a mass chromatogram. A “mass chromatogram” is a representation of mass spectrometry data as a chromatogram. Typically, the x-axis represents time and the y-axis represents signal intensity. In one aspect the mass chromatogram is a total ion current (TIC) chromatogram. In another aspect, the mass chromatogram is a base peak chromatogram. In other embodiments, the mass chromatogram is a selected ion monitoring (SIM) chromatogram. In yet another embodiment, the mass chromatogram is a selected reaction monitoring (SRM) chromatogram. In one embodiment, the mass chromatogram is an extracted ion chromatogram (EIC). In an EIC, a single feature is monitored throughout the entire run. The total intensity or base peak intensity within a mass tolerance window around a particular analyte's mass-to-charge ratio is plotted at every point in the analysis. The size of the mass tolerance window typically depends on the mass accuracy and mass resolution of the instrument collecting the data. As used herein, the term “feature” refers to a single small metabolite, or a fragment of a metabolite. In some embodiments, the term feature may also include noise upon further investigation.

In some embodiments, detection of the presence of a metabolite involves detection of signal intensity. This, in turn, can reflect the quantity and character of a biomarker. For example, in certain embodiments, the signal strength of peak values from spectra of a first sample and a second sample can be compared (e.g., visually, by computer analysis etc.) to determine the relative amounts of particular metabolites. Software programs such as the Biomarker Wizard program (Ciphergen Biosystems, Inc., Fremont, Calif.) can be used to aid in analyzing mass spectra. The mass spectrometers and their techniques are well known.

A person skilled in the art understands that any of the components of a mass spectrometer, e.g., desorption source, mass analyzer, detect, etc., and varied sample preparations can be combined with other suitable components or preparations described herein, or to those known in the art. For example, in some embodiments a control sample may contain heavy atoms, e.g. ¹³C, thereby permitting the test sample to be mixed with the known control sample in the same mass spectrometry run.

In some embodimentd, a laser desorption time-of-flight (TOF) mass spectrometer is used. In laser desorption mass spectrometry, a substrate with a bound marker is introduced into an inlet system. The marker is desorbed and ionized into the gas phase by laser from the ionization source. The ions generated are collected by an ion optic assembly, and then in a time-of-flight mass analyzer, ions are accelerated through a short high voltage field and let drift into a high vacuum chamber. At the far end of the high vacuum chamber, the accelerated ions strike a sensitive detector surface at a different time. Since the time-of-flight is a function of the mass of the ions, the elapsed time between ion formation and ion detector impact can be used to identify the presence or absence of molecules of specific mass to charge ratio. In one embodiment of the invention, levels of metabolites are detected by MALDI-TOF mass spectrometry.

Methods of detecting metabolites also include the use of surface plasmon resonance (SPR). The SPR biosensing technology has been combined with MALDI-TOF mass spectrometry for the desorption and identification of metabolites.

Data for statistical analysis can be extracted from chromatograms (spectra of mass signals) using software for statistical methods known in the art. “Statistics” is the science of making effective use of numerical data relating to groups of individuals or experiments. Methods for statistical analysis are well-known in the art. In one embodiment a computer is used for statistical analysis. In one embodiment, the Agilent MassProfiler or MassProfilerProfessional software is used for statistical analysis. In another embodiment, the Agilent MassHunter software Qual software is used for statistical analysis. In other embodiments, alternative statistical analysis methods can be used. Such other statistical methods include the Analysis of Variance (ANOVA) test, Chi-square test, Correlation test, Factor analysis test, Mann-Whitney U test, Mean square weighted derivation (MSWD), Pearson product-moment correlation coefficient, Regression analysis, Spearman's rank correlation coefficient, Student's T test, Welch's T-test, Tukey's test, and Time series analysis.

In various embodiments, signals from mass spectrometry are transformed in different ways to improve the performance of the method. Either individual signals or summaries of the distributions of signals (such as mean, median or variance) can be so transformed. Possible transformations include taking the logarithm, taking some positive or negative power, for example the square root or inverse, or taking the arcsin (Myers, Classical and Modern Regression with Applications, 2nd edition, Duxbury Press, 1990).

In some embodiments, the ability to quantitate the amount of a metabolite (or multiple metabolite) in a biological sample from a subject allows a clinician to make assessments regarding the condition of the subject, to further provide a diagnosis or prognosis for the subject, and/or to further recommend or administer a treatment course of action. Thus, according to another aspect of the present invention there is provided a method of characterizing the risk of mortality a subject suffering from COVID-19 (e.g., severe COVID-19) and/or severe respiratory insufficiency (e.g., severe respiratory insufficiency) comprising determining the abundance of at least one metabolite in a biological sample from the subject (e.g., stool sample, rectal swab, etc.), comparing the quantity to a control or threshold value, and providing a prognosis regarding the likelihood of survival/mortality and/or a treatment course of action for the subject. Once the level of one or more metabolites is measured by the techniques described herein and/or understood in the field, it is typically compared to a level of that metabolite in a control subject or a threshold value. In some embodiments, if the metabolite level is above or below the control or threshold, determinations about the state or the subject can be inferred or concluded. In embodiments in which the levels of multiple metabolites are measured to provide a prognosis or to determine a treatment course of action, an algorithm may be employed to combine the level of the multiple biomarkers, and/or their levels relative to individual thresholds, into a single prognosis or treatment course of action. In some embodiments, a score is provided for each metabolite, based on the abundance of the metabolite in the sample relative to a control or threshold value. In some embodiments, the combination of scores from multiple metabolites is used to provide a prognosis and/or determine a treatment course of action. In some embodiments, if a metabolite level is above a threshold it is given a first score (e.g., 1 or 0) and if the metabolite level is below a threshold it is given a second score (e.g., 0 or 1). In some embodiments, a metabolite level is scored based on how far above or below the metabolite level is relative to a threshold (e.g., scored 0-100). In some embodiments, the scores of multiple metabolites are combined to provide a prognosis (e.g., likelihood of survival/mortality (e.g., qualitative (e.g., high, intermediate, or low), percentage (e.g., 50%, 60%, 70%, 80%, 90%, etc.), etc.)). In some embodiments, the combined score of multiple metabolites (e.g., based on comparison to thresholds) is compared to a ‘panel threshold’ or multiple panel thresholds to classify the subject and the likelihood of survival/mortality. In some embodiments, comparison to a panel threshold or multiple panel thresholds allows for stratification (e.g., qualitative or quantitative) of the likelihood of survival/mortality of a subject. In some embodiments, examples of a qualitative likelihood of mortality are low risk, intermediate risk, high risk, severe risk, etc. In some embodiments, examples of a quantitative likelihood of mortality are 10%, 20%, 30%, 40%, 50%, 60%, 70%, 80%, 90%, 95%, etc. Individual and/or combined metabolite scores, based on comparison to controls or threshold values, can be determined and combined in any suitable manner to achieve a statistically relevant prognosis. The scope herein is not limited by the various ways of comparing metabolite levels to thresholds and combining them to produce a prognosis and/or treatment course of action.

Embodiments herein find use in providing a survival/mortality prognosis for subjects suffering from COVID-19 and/or respiratory insufficiency.

Coronavirus disease 2019 (COVID-19) is a condition resulting from infection of a subject (e.g., human) by the severe acute respiratory syndrome coronavirus 2 (SARS-CoV-2). Patients with SARS-CoV-2 infection (e.g., suffering from COVID-19) can experience a range of clinical manifestations, from no symptoms to critical illness. In general, subjects (e.g., adults) with SARS-CoV-2 infection can be grouped into the following severity of illness categories; however, the criteria for each category may overlap or vary across clinical guidelines and clinical trials, and a patient's clinical status may change over time:
Asymptomatic or Presymptomatic Infection: Individuals who test positive for SARS-CoV-2 using a virologic test (i.e., a nucleic acid amplification test [NAAT] or an antigen test) but who have no symptoms that are consistent with COVID-19.
Mild Illness: Individuals who have any of the various signs and symptoms of COVID-19 (e.g., fever, cough, sore throat, malaise, headache, muscle pain, nausea, vomiting, diarrhea, loss of taste and smell) but who do not have shortness of breath, dyspnea, or abnormal chest imaging.
Moderate Illness: Individuals who show evidence of lower respiratory disease during clinical assessment or imaging and who have an oxygen saturation (SpO₂)≥94% on room air at sea level.
Severe Illness: Individuals who have SPO₂<94% on room air at sea level, a ratio of arterial partial pressure of oxygen to fraction of inspired oxygen (PaO2/FiO2)<300 mm Hg, a respiratory rate >30 breaths/min, or lung infiltrates >50%.
Critical Illness: Individuals who have respiratory failure, septic shock, and/or multiple organ dysfunction.
   In some embodiments, methods and biomarker panels herein find use in providing a prognosis for a subject suffering from moderate to critical COVID-19. In some embodiments, such a prognosis provides a clinician with additional information to select and administer a treatment course of action.

In some embodiments, a subject suffers from respiratory failure, respiratory insufficiency, respiratory disease, cytokine storm, or other conditions that may be related to or independent of COVID-19.

In some embodiments, a biological sample is obtained from a subject. In some embodiments, the biological sample is tested for biomarkers (e.g., microbiomic, metabolomic, etc.), and an analysis is performed to provide a clinician with a prognosis and treatment course of action. In some embodiments, a biological sample is a fecal sample. The fecal sample can be collected from feces after defecation, such as a stool sample, or from feces obtained directly from the rectum of a subject. In some embodiments, a biological sample is gastrointestinal lavage fluid (GLF). In some embodiments, the biological sample is tested directly for biomarkers (e.g., microbiomic, metabolomic, etc.). In some embodiments, the biological sample is treated prior to analysis. For example, a sample may be fractionated, centrifuged, liquefied, diluted, etc. prior to analysis.

In some embodiments, the methods herein provide decision-making information regarding a treatment course of action. In some embodiments, methods herein comprise a step of administering a treatment for COVID-19, respiratory insufficiency, and/or any symptoms or conditions related thereto. Suitable treatments that may be administered or the doses adjusted based on the results of the biomarker testing herein include remdesivir, dexamethasone (or alternative corticosteroids), baricitinib, heparin, tofacitinib, tocilizumab, and sarilumab.

In some embodiments, methods herein identify subjects are risk (e.g., low, moderate, high, severe, etc.) of progressive respiratory failure. In some embodiments, subjects identified as being at high or severe risk of respiratory failure are administered aggressive treatment including but not limited to antivirals and anti-inflammatory agents (e.g., steroids). In some embodiments, high or severe risk are administered microbiome augmentation with prebiotics, probiotics (i.e., live biotherapeutic products) or microbiota-derived metabolites. In some embodiments, a subject high or severe risk is administered metabolites identified herein as correlating with survival of COVID-19 and/or respiratory insufficiency. In some embodiments, a subject at high or high or severe risk is administered bacteria identified herein as correlating with survival of COVID-19 and/or respiratory insufficiency.

In some embodiments, a subject is administered metabolites and/or beneficial bacteria based on the levels determined in the tested described herein. For example, a subject determined to be low in deoxycholic acid, isodeoxycholic acid, lithocholic acid, and/or desaminotyrosine may be administered one or more of deoxycholic acid, isodeoxycholic acid, lithocholic acid, and/or desaminotyrosine. In some embodiments, a subject determined to be low in beneficial bacteria may be administered basterai of the families Bacteroidaceae and/or Lachnospiraceae.

### EXPERIMENTAL

### Material and Methods

### Study Design & Patient Enrollment:

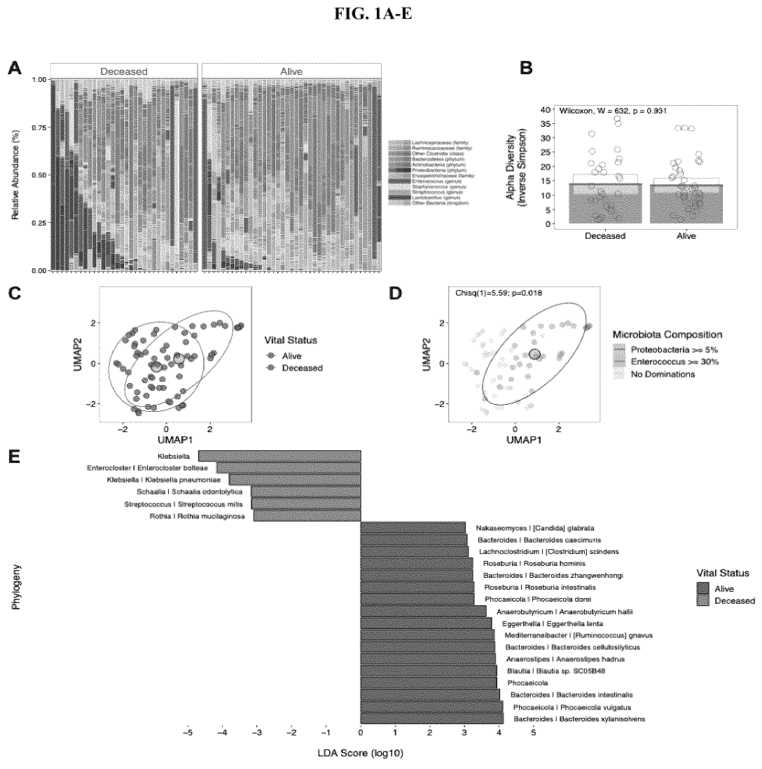

Patients with COVID-19 associated respiratory failure or shock admitted to the medical ICU were included in the study. For the purposes of the inclusion criteria, respiratory failure was defined by the receipt of non-invasive positive pressure ventilation (NIPPV), high flow nasal cannula (HFNC), or invasive mechanical ventilation. Shock was defined by the receipt of vasoactive medications. Exclusion criteria included: age <18 years, pregnancy, and prior cardiac arrest during admission of interest. COVID-19 diagnosis was confirmed by reverse transcriptase-polymerase chain reaction of nasal pharyngeal swabs. This project received institutional review board (IRB) approval from the University of Chicago (20-1102). Informed consent was obtained from the patient or surrogate decision makers prior to enrollment. Enrollment began in September 2020 and concluded in May of 2021. Patient enrollment is described in Supplement FIG. 1. Patients were followed up to 1 year following study completion by chart review and telephone.

### Specimen Collection and Analysis:

Fecal samples were collected as soon as possible following ICU admission. Samples which were collected within the first 72 hours of ICU hospitalization at University of Chicago were included in this analysis. This time frame was chosen to focus the study on whether the fecal microbiota in the early course of illness influences clinical trajectories and outcomes.

### Metagenomic Analyses:

Fecal samples underwent metagenomic shotgun DNA sequencing. Samples underwent mechanical disruptions with a bead beater (BioSpec Product) and were further purified with QIAamp mini spin columns (Qiagen). Purified DNA was quantified with a Qubit 2.0 fluorometer and sequenced on the Illumina HiSeq platform. Fecal samples from clinical patients were prepared in batches of 60-72 in paired-end (PE) libraries with insert size around 350 bp for each sample. High-throughput sequencing on Illumina NextSeq 500/NovaSeq 6000 produced around 7 to 8 million PE reads per sample with read length of 149/159 bp. Adapters were trimmed off from the raw reads, and their quality was assessed and controlled using Trimmomatic (v.0.39) (Ref. 36; incorporated by reference in its entirety). Reads mapped to the human genome were be identified and removed by kneaddata (v0.7.10). Microbial reads were assembled using MEGAHIT (v1.2.9) (Ref. 37; incorporated by reference in its entirety) and genes were called by prodigal and annotated with prokka (v.1.14.6) (Ref. 38; incorporated by reference in its entirety). The translated proteins for each fecal microbiome were functionally profiled by eggnog mapper (2.0.1b) (Ref. 39; incorporated by reference in its entirety) against their default precomputed orthologous groups and phylogenies from the EggNOG database including presence/absence of KOs (KEGG Orthologies) from the KEGG database (Ref. 40; incorporated by reference in its entirety). Genes encoding for antibiotic resistance (Ref. 41; incorporated by reference in its entirety) and bacteriocins were queried against high-quality shotgun reads using DIAMOND (v2.0.4.) with a filter threshold of ≥80% identity and ≥80% protein coverage. Reads are reported in reads per million normalized by the length of the gene, in kilobases (RPKM). Toxins/hemolysins/cytolysins genes were obtained from the EggNOGG database alignment and returned as presence/absence of a KO. Taxonomy was profiled using Kraken2 on PATRIC v3.5.0. Alpha diversity of fecal samples was calculated using the Inverse Simpson Index.

### Metabolomic Analyses

Three short chain fatty acids (butyrate, acetate, and propionate) and succinate were derivatized with pentafluorobenzyl bromide (PFBBr) and analyzed via negative ion collision induced-gas chromatography-mass spectrometry ([-]CI-GC-MS, Agilent 8890). Eight bile acids (primary: cholic acid; conjugated primary: glycocholic acid, taurocholic acid; secondary: deoxycholic acid, lithocholic acid, isodeoxycholic acid, alloisolithocholic acid and 3-oxolithocholic acid) (g/mL) were quantified by negative mode liquid chromatography-electrospray ionization-quadrupole time-of-flight-MS ([-]LC-ESI-QTOF-MS, Agilent 6546). Desaminotyrosine and indole-3-carboxaldehyde were analyzed via UPLC-QqQ LC-MS (μM). Ninety-two total additional compounds were relatively quantified using normalized peak areas relative to internal standards. Additional details regarding metabolite analysis can be found in the methods supplement document. Insufficient sample precluded metabolite analysis of 7 patients.

### Development of the Microbiome Metabolite Profile:

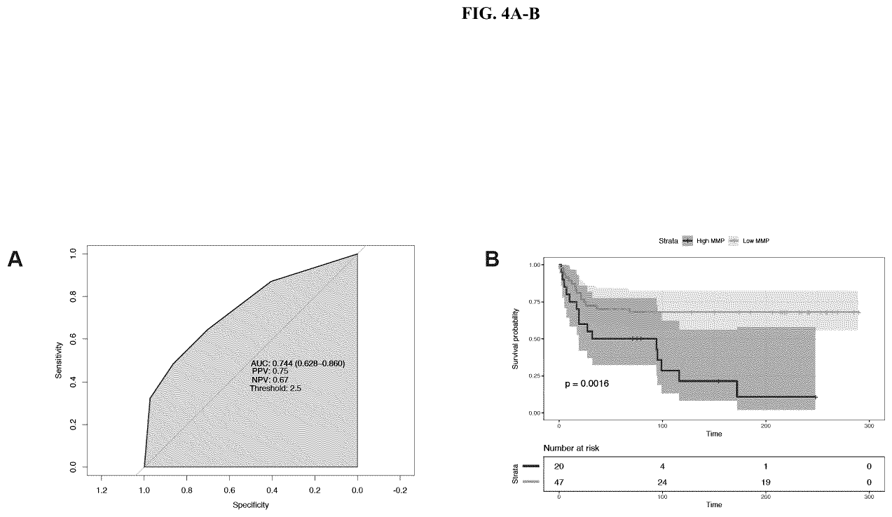

The microbiome metabolite profile (MMP), was developed as an aggregate of selected metabolic features of the microbiome that might reflect its functional potential using the R programming language (v 4.1.1). Metabolites were down-selected using both the results from the volcano analysis (FIG. 3A) as well as metabolites that maintained biological plausibility (i.e. deoxycholic acid, isodeoxycholic acid, lithocholic acid, and desaminotyrosine). Optimized thresholds for these four compounds (concentrations) were determined using the Youden Index (cutpointr:cutpointr, v1.1.1) which individually selects the metabolite concentration that optimizes sensitivity and specificity, where outcomes were binary (alive/deceased; Table 3) (Ref. 42; incorporated by reference in its entirety). If concentrations were less than the optimized thresholds, one point was assigned to that compound, where scores that correlate to survival were zero and scores that correlate to death were one; a minimum of 0 points and a maximum of 4 points could be assigned. To assess the model, a receiver operator characteristic curve (ROC) analysis was performed (FIG. 4A). In addition to the AUC score, the optimized threshold, positive predicted value (PPV) and negative predicted value (NPV) were also calculated.

### Clinical Data:

Clinical data was obtained through a data extraction procedure of the electronic medical record and confirmed by manual chart review or by manual chart review alone.

### Statistical Analysis:

All statistical analyses were conducted using the R programming language (version 4.1.1). Adjusted p-values of the tests were considered to be statistically significant for all analyses conducted if p≤0.05. In some instances, unadjusted p-values were also displayed. Continuous variables were compared between the survival groups using Wilcoxon rank-sum test (rstatix::wilcox_test) and p-values were adjusted following the Benjamini-Hochberg method (rstatix:: adjust_pvalue). Categorial variables were compared using the chi-squared test (chisq.test). Kaplan-Meier curves for survival endpoints were generated as well as stratified by selected risk factors such as protobacteria abundance (normal/abnormal; survival::Surv, survfit, ggsurvplot) and a log-rank test was used to assess significant differences (glm). A Cox proportional hazards regression model for mortality and a relative risk regression model for progression of respiratory failure indicator were used to estimate the effects of microbiome and metabolites adjusting for known risk factors (survival::coxph).

### Metabolomic Sample Preparation and Analyses

Extraction solvent (80% methanol spiked with internal standards and stored at −80° C.) was added to pre-weighed fecal samples at a ratio of 100 mg of material/mL of extraction solvent in beadruptor tubes. Samples were homogenized at 4° C. set at 1.6 m/s with 6 thirty-second cycles, 5 seconds off per cycle. Samples were then centrifuged at −10° C., 20,000×g for 15 min and the supernatant was used for subsequent metabolomic analysis. Short chain fatty acids were derivatized as described by Haak et al. with the following modifications.⁴³ The metabolite extract (100 μL) was added to 100 μL of 100 mM borate buffer (pH 10), 400 μL of 100 mM pentafluorobenzyl bromide in acetonitrile, and 400 μL of n-hexane. Samples were heated in a thermomixer to 65° C. for 1 hour while shaking at 1, 300 rpm. After cooling to room temperature, samples were centrifuged at 4° C., 2,000×g for 5 min, allowing phase separation. The hexanes phase (100 μL) (top layer) was transferred to an autosampler vial containing a glass insert and the vial was sealed. Another 100 μL of the hexanes phase was diluted with 900 μL of n-hexane in an autosampler vial. Concentrated and diluted samples were analyzed using a gas chromatography-mass spectrometry (GC-MS, Agilent 7890A GC system, Agilent 5975C MS detector) operating in negative chemical ionization mode, using a HP-5MSUI column (30 m×0.25 mm, 0.25 μm; Agilent Technologies), methane as the reagent gas (99.999% pure) and 1 μL split injection (1:10 split ratio). Oven ramp parameters: 1 min hold at 60° C., 25° C. per min up to 300° C. with a 2.5 min hold at 300° C. Inlet temperature was 280° C. and transfer line was 310° C. A 10-point calibration curve was prepared with acetate (100 mM), propionate (25 mM), butyrate (12.5 mM), and succinate (50 mM), with 9 subsequent 2× serial dilutions. Data analysis was performed using MassHunter Quantitative Analysis software (version B.10, Agilent Technologies) and confirmed by comparison to authentic standards. Normalized peak areas were calculated by dividing raw peak areas of targeted analytes by averaged raw peak areas of internal standards. Bile acids were analyzed using reverse phase liquid chromatography-mass spectrometry (RPLC-MS). The metabolite extract (75 μL) was dried down under a nitrogen stream at 30 L/min (top) 1 L/min (bottom) at 30° C. Samples were resuspended in 50:50 water:methanol (750 μL). Vials were added to a thermomixer to resuspend analytes at 4° C., 1000 rpm for 15 min with an infinite hold at 4° C. Samples were then transferred to microcentrifuge tubes and centrifuged at 4° C., 20,000×g for 15 min to remove insoluble debris. The supernatant (700 μL) was transferred to a fresh vial. Samples were analyzed on a liquid chromatography system (Agilent 1290 infinity II) coupled to a quadrupole time-of-flight (QTOF) mass spectrometer (Agilent 6546), operating in negative mode, equipped with an Agilent Jet Stream Electrospray Ionization source. The sample (5 μL) was injected onto an XBridge® BEH C18 Column (3.5 μm, 2.1×100 mm; Waters Corporation, PN) fitted with an XBridge® BEH C18 guard (Waters Corporation, PN) at 45° C. Elution started with 72% A (Water, 0.1% formic acid) and 28% B (Acetone, 0.1% formic acid) with a flow rate of 0.4 mL/min for 1 min and linearly increased to 33% B over 5 min, then linearly increased to 65% B over 14 min. Then the flow rate was increased to 0.6 mL/min and B was increased to 98% over 0.5 min and these conditions were held constant for 3.5 min. Finally, re-equilibration at a flow rate of 0.4 mL/min of 28% B was performed for 3 min. The electrospray ionization conditions were set with the capillary voltage at 3.5 kV, nozzle voltage at 2 kV, and detection window set to 100-1700 m/z with continuous infusion of a reference mass (Agilent ESI TOF Biopolymer Analysis Reference Mix) for mass calibration. A ten-point calibration curve was used for quantitation. Data analysis was performed using MassHunter Profinder Analysis software (version B.10, Agilent Technologies) and confirmed by comparison with authentic standards. Normalized peak areas were calculated by dividing raw peak areas of targeted analytes by averaged raw peak areas of internal standards. Quantitation of desaminotyrosine from extracts was completed with reverse phase separation of samples and analysis by triple quadrupole mass spectrometry (RPLC-QQQ). Samples were analyzed on an Agilent 1290 infinity II liquid chromatography system coupled to an Agilent 6470 triple quadrupole mass spectrometer, operating in negative mode, equipped with an Agilent Jet Stream Electrospray Ionization source. Each sample (2 μL) was injected into an Acquity UPLC HSS PFP column, 1.8 μm, 2.1×100 mm (Waters; 186005967) equipped with an Acquity UPLC HSS PFP VanGuard Pre-column, 100 Å, 1.8 μm, 2.1 mm×5 mm (Waters; 186005974) at 45° C. Mobile phase A was 0.1% formic acid in water and mobile phase B was 0.1% formic acid in 95:5 acetonitrile:water. The flow rate was set to 0.5 mL/min starting at 0% B held constant for 3 min, then linearly increased to 50% over 5 min, then linearly increased to 95% B over 1 min, and held at 100% B for the next 3 min. Mobile phase B was then brought back down to 0% over 0.5 min and held at 0% for re-equilibration for 2.5 min. The QQQ electrospray conditions were set with capillary voltage at 4 kV, nozzle voltage at 500 V, and Dynamic MRM was used with cycle time of 500 ms. Transitions were monitored in negative mode, and the transition for desaminotyrosine was 165.17 m/z to m/z 93.1. An 11-point calibration curve (ranging from 0.98 nM to 1 mM) was prepared using a desaminotyrosine standard. Data analysis was performed using MassHunter Quant software (version B.10, Agilent Technologies) and confirmed by comparison with authentic standards. Normalized peak areas were calculated by dividing raw peak areas of targeted analytes by averaged raw peak areas of internal standard.

### Results

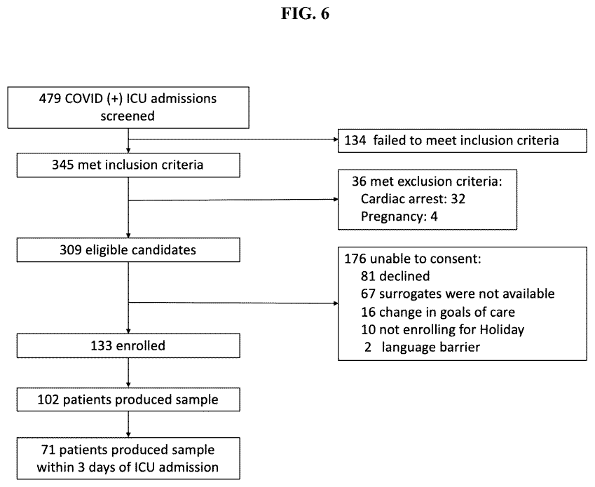

Between September 2020 and May 2021, 102 patients with COVID-19 were enrolled in a fecal collection protocol upon admission to the Medical Intensive Care Unit (ICU) at the University of Chicago Medical Center (FIG. 6). Of these, 71 patients produced a fecal sample within 72 hours of enrollment (mean time of collection 24.7 hours). Patient baseline information, clinical characteristics and antibiotic/antiviral treatment are stratified by mortality in Table 1. There were no significant differences between patients who survived versus died in terms of race, gender, diabetes, age, body mass index, hypertension, or chronic kidney disease. The two groups did not significantly differ in terms of treatment with antibiotics or COVID-19 specific therapies, such as steroids or remdesivir.

TABLE 1
Patient Characteristics Stratified by Mortality

| | Alive | | Deceased | | |
|---|---|---|---|---|---|
| n | 39 | | 32 | | p |
| Baseline Characteristics | | | | | |
| Race (%) | | | | | 0.366 |
| Asian/Mideast Indian | 2 | (6.2) | 0 | (0.0) | |
| Black/African-American | 25 | (64.1) | 19 | (59.4) | |
| Hispanic | 3 | (7.7) | 6 | (18.8) | |
| White | 9 | (23.1) | 7 | (21.9) | |
| Male (%) | 19 | (48.7) | 18 | (60.0) | 0.491 |
| Age (median [IQR]) | 58.97 | [51.91, 69.01] | 66.21 | [56.56, 72.66] | 0.166 |
| Body Mass Index (median [IQR]) | 31.51 | [27.96, 38.78] | 30.73 | [26.83, 34.08] | 0.432 |
| Hypertension (%) | 23 | (59.0) | 23 | (71.9) | 0.377 |
| Hyperlipidema (%) | 12 | (30.8) | 11 | (34.4) | 0.946 |
| Diabetes (%) | 13 | (33.3) | 12 | (37.5) | 0.908 |
| Cancer (%) | 6 | (15.4) | 3 | (9.4) | 0.69 |
| Chronic Kidney Disease (%) | 3 | (7.7) | 8 | (25.0) | 0.094 |
| Clinical Characteristics | | | | | |
| Charison Comorbidity Index (median [IQR]) | 3.00 | [2.00, 4.50] | 4.00 | [2.00, 5.00] | 0.551 |
| SOFA Score (median [IQR]) | 5.00 | [4.00, 9.00] | 9.00 | [8.00, 9.00] | <0.001 |
| APACHE Score (median [IQR]) | 18.00 | [13.00, 22.50] | 23.00 | [19.00, 30.25] | 0.002 |
| Days From Symptom Onset (median [IQR]) | 5.00 | [3.00, 7.00] | 5.00 | [2.75, 8.00] | 0.907 |
| Admission (%) | | | | | 0.229 |
| Emergency Department | 25 | (64.1) | 14 | (43.8) | |
| Hospital Medicine | 8 | (20.5) | 10 | (31.2) | |
| Outside Hospital | 6 | (15.4) | 8 | (25.0) | |
| Medication Administration | | | | | |
| Antivirals (%) | | | | | |
| Remdesivir Treatment | 28 | (71.8) | 22 | (68.8) | 0.985 |
| Steroid Treatment | 28 | (71.8) | 23 | (71.9) | 1 |
| Antibiotics (%) | | | | | |
| Betalactams | 8 | (20.5) | 5 | (15.6) | 0.825 |
| Levofloxicin | 1 | (2.6) | 0 | (0.0) | 1 |
| Vancomycin | 9 | (23.1) | 13 | (40.6) | 0.116 |
| Metronidazole | 3 | (7.7) | 4 | (12.5) | 0.782 |
| Macrolides | 8 | (20.5) | 6 | (18.8) | 1 |
| Doxycycline | 5 | (12.8) | 1 | (3.1) | 0.302 |
| Trimethoprim-Sulfamethoxazole | 5 | (12.8) | 2 | (6.2) | 0.6 |
| Aminoglycosides | 1 | (2.6) | 0 | (0.0) | 1 |

Description of patient baseline demographics, past medical history, severity of illness and relevant medications. Categorical Variables compared with Chi-Square. Continuous variables were compared Wilcoxon Rank Sum Test. Adequate treatment with COVID-19 specific therapy included at least three consecutive days of therapy during index hospitalization. Sufficient total dose of steroids was the equivalent of 18 mg of dexamethasone and 400 mg of Remdesivir. Only antibiotics received 72 hours prior to fecal specimen collection are represented.

Microbiome compositions in each fecal sample, stratified by mortality, are shown in FIG. 1A and reveal higher densities of proteobacteria in patients who died of severe COVID-19 (Table 2). While microbiome alpha diversity, as determined by inverse Simpson, did not differ between patients who survived versus those who died (10.7 vs 10.8, p=0.93) (FIG. 1B, Table 2), dimension reduction of microbiome compositions by Uniform Manifold Approximation and Projection (UMAP) demonstrated distinct clustering of patients who died versus patients who recovered from COVID-19 (FIG. 1C), with statistically significant overlap of Proteobacteria dominated clusters with mortality (FIG. 1D, χ²(1, N=71)=5.59, p=0.018). Linear discriminant analysis effect size (LEfSe) using shotgun metagenomics-based taxonomy data indicated survival was associated with increased representation of obligate anaerobic bacterial species belonging to the Bacteroidaceae and Lachnospiraceae families while mortality was associated with expansion of Enterobacteriaceae (FIG. 1E). Furthermore, Proteobacteria expansion (relative abundance of >5%) was significantly associated with higher mortality (Table 2, p=0.035).

TABLE 2

| | Alive | | Deceased | | |
|---|---|---|---|---|---|
| n | 39 | | 32 | | p |
| Microbiologic Characteristics | | | | | |
| Diversity: (median [IQR]) | | | | | |
| Inverse Simpson | 10.77 | [7.53, 18.05] | 10.80 | [5.74, 20.70] | 0.926 |
| Relative Abundance: (% of group) | | | | | |
| Proteobacteria Domination | 9 | (23.1) | 16 | (50.0) | 0.035 |
| Enterococcus Domination | 3 | (7.7) | 3 | (9.4) | 1 |
| Metabolites | | | | | |
| Desaminotyrosine (μM) | 28.10 | [22.74, 46.94] | 22.63 | [21.00, 32.30] | 0.028 |
| Lithocholic acid (μg/mL) | 61.61 | [4.30, 252.61] | 9.76 | [0.24, 60.07] | 0.008 |
| Deoxycholic acid (μg/mL) | 74.09 | [18.61, 246.34] | 10.68 | [0.49, 62.50] | 0.003 |
| Isodeoxycholic acid (μg/mL) | 7.60 | [0.70, 25.09] | 2.89 | [0.06, 10.93] | 0.038 |
| Cholic acid (μg/mL) | 16.21 | [1.4, 215.59] | 7.92 | [0.79, 88.18] | 0.213 |
| Glycocholic acid (μg/mL) | 0.43 | [0.18, 2.83] | 0.45 | [0.03, 11.78] | 0.512 |
| 3-oxolithocholic acid (μg/mL) | 4.71 | [0.64, 54.90] | 1.88 | [0.06, 14.03] | 0.086 |
| Alloisolithocholic acid (μg/mL) | 0.44 | [0.00, 2.79] | 0.20 | [0.00, 6.18] | 0.482 |
| Taurocholic acid (μg/mL) | 0.55 | [0.22, 2.23] | 0.30 | [0.08, 24.06] | 0.391 |
| Butyrate (mmol) | 0.51 | [0.20, 2.39] | 0.29 | [0.06, 0.97] | 0.286 |
| Propionate (mmol) | 1.55 | [0.47, 4.94] | 0.75 | [0.19, 1.62] | 0.169 |
| Acetate (mmol) | 3.87 | [2.29, 20.44] | 3.13 | [1.17, 11.23] | 0.175 |
| Microbiome Metabolic Profile (median [IQR]) | 0.0 | [0.00, 2.00] | 2.00 | [1.00, 3.00] | <0.001 |

Characteristics of the fecal microbiome stratified by mortality.
Categorical Variables compared with Chi-Square.
Continuous variables were compared Wilcoxon Rank Sum Test.
Unadjusted p-values are presented.

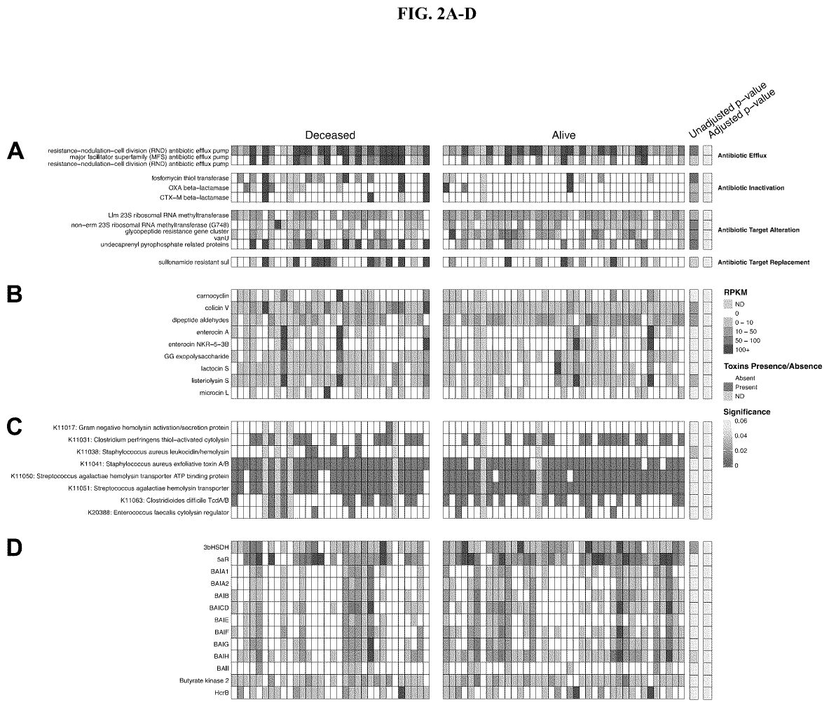

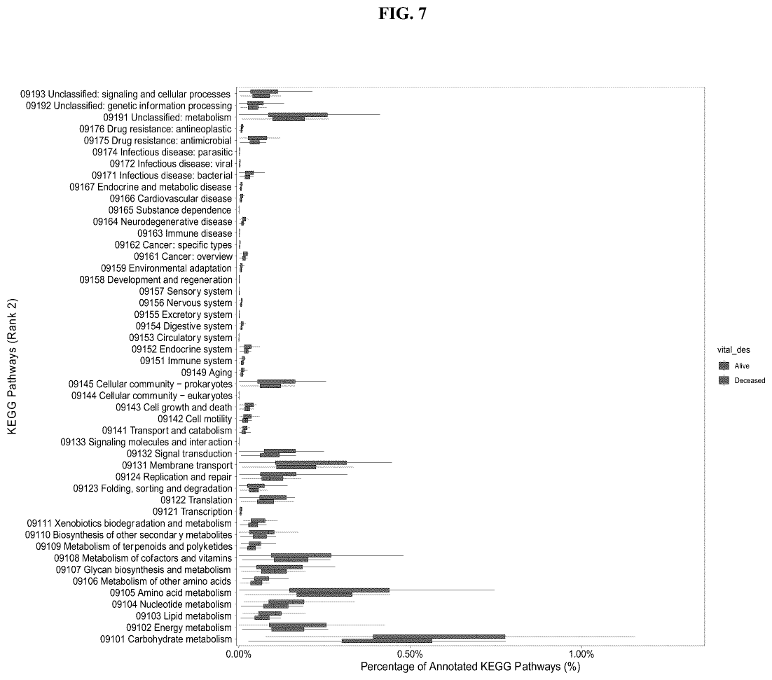

Although microbiota compositions differed between COVID-19 patients who survived versus died of COVID-19, comparison of KEGG metabolic pathways did not identify significant differences between the two groups (FIG. 7). Because most metabolic pathways included in this comparison contribute to general bacterial physiology, and thus might conceal less prevalent pathways that contribute to COVID-19 pathogenesis, experiments were conducted during development of embodiments herein that focused on genes encoding bacterial antibiotic resistance, bacteriocins and toxins/hemolysins/cytolysins (FIG. 2A-C). After correcting for multiple comparisons, statistically significant differences were not identified between the two groups. However, there was a trend towards increased representation of antibiotic-resistance genes in the patient group that died of COVID-19. Because secondary bile acids and butyrate have been shown to be immunomodulatory, the frequencies of genes encoding 3βHydroxysteroid dehydrogenase (3βHSDH), 5αReductase (5AR), the Bai operon and Butyrate Kinase were quantified, but significant differences between the two patient groups were not detected (FIG. 2D).

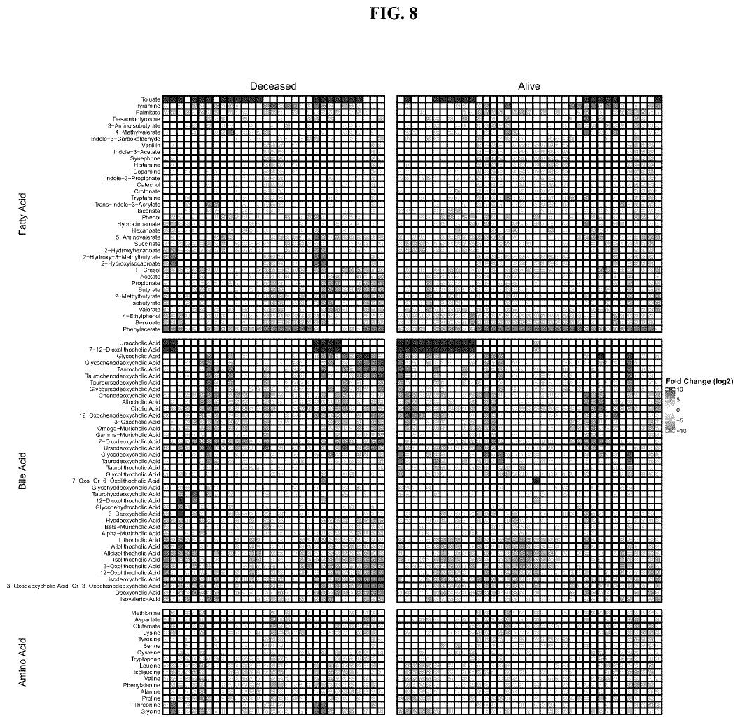

Although the metabolic output of the intestinal microbiota is determined by its composition, other factors, such as diet and the host's state of immune activation and inflammation, also impact metabolite production. Thus, compositionally similar microbiota can establish distinct fecal metabolomes, resulting from changes in diet, medications and/or the host's state of immune activation (Ref. 25; incorporated by reference in its entirety). To determine whether fecal metabolomes differed between patients who survived or died from COVID-19, GC- and LC-MS was performed on fecal samples to quantify a range of fatty acids, amino acids, bile acids and other metabolites known to be produced by commensal bacteria. Differences in representation of 92 metabolites are presented in a heat map of normalized values and are expressed as fold changes relative to the mean value for all samples (FIG. 8). A volcano plot more clearly identifies metabolites associated with survival, including secondary bile acids, short chain fatty acids and desaminotyrosine (FIG. 3A). Deoxycholic acid, lithocholic acid, isodeoxycholic acid and desaminotyrosine were each associated with survival (FIGS. 3A and B, FIG. 8). Univariate analyses demonstrated significant associations between lithocholate (p=0.008), deoxycholate (p=0.003), isodeoxycholate (p=0.038), and desaminotyrosine (p=0.028) (FIG. 3B, Table 2) and survival of COVID-19. Butyrate, acetate and propionate concentrations, while reduced in COVID-19 patients who died, did not achieve significance (FIG. 8, Table 2).

Given the parallel and mechanistically distinct contributions of intestinal microbes and their metabolites to immune modulation and inflammatory responses, a profile was developed (referred to herein as the Microbiome Metabolite Profile (MMP)) to more comprehensively associate the microbiome's function with clinical outcomes. The components of the MMP, deoxycholic acid, lithocholic acid, isodeoxycholic acid and desaminotyrosine, were selected based on association with survival and their plausible immune regulatory and antiviral roles during SARS-CoV-2 infection (Table 3). With respect to mortality, the MMP demonstrated an AUC=0.74 (CI=0.628-0.860) with negative predictive value of 0.67 and positive predictive value of 0.75 (FIG. 4A). Kaplan-Meier survival curves demonstrate 32.1% mortality in the low MMP (MMP=0-1) group compared to 89.3% mortality in high MMP (MMP=2-4) group (p=0.005) (FIG. 4B). To test for independent association with mortality, the MMP, as the sole evaluator of microbiome health, was included in a Cox proportional hazard model along with other variables with univariable p-value <0.3. This model demonstrated that at any point in the study, patients with a high MMP score were 76% more likely to die than patients with a low MMP score (HR=1.76, CI=1.24-2.49, p=0.002) (Table 4).

TABLE 3
Microbiome Metabolite Profile

| | Points | | |
|---|---|---|---|
| Compound | | 0 | 1 |

| | | | | |
|---|---|---|---|---|
| Deoxycholic Acid | ≥89.92 | (μM) | <89.92 | (μM) |
| Isodeoxycholic Acid | ≥0.97 | (μM) | <0.97 | (μM) |
| Lithocholic Acid | ≥258.25 | (μM) | <258.25 | (μM) |
| Desaminotyrosine | ≥21.31 | (μM) | <21.31 | (μM) |

Quantitated metabolomic compounds (μM) and threshold values developed for the Microbiome Metabolite Profile.
Variables chosen based on biologic plausibility and statistical significance.
Higher scores indicated microbiome dysfunction.

TABLE 4
Cox Proportional Hazards Regression Model for Mortality

| | HR¹ | 95% CI¹ | p-value |
|---|---|---|---|
| Characteristic | | | |
| Age | 1.05 | 1.01, 1.09 | 0.005 |
| Chronic Kidney Disease | 1.05 | 0.40, 2.74 | >0.9 |
| SOFA Score | 1.50 | 1.28, 1.77 | <0.001 |
| Vancomycin Adminstration | 0.33 | 0.13, 0.84 | 0.021 |
| Admission Location | | | |
| ED | — | — | |
| Hospital Medicine | 1.56 | 0.60, 4.05 | 0.4 |
| OSH | 0.15 | 0.03, 0.78 | 0.024 |
| Microbiome Metabolic Profile | 1.76 | 1.24, 2.49 | 0.002 |

¹HR = Hazard Ratio, CI = Confidence Interval
Cox Proportional Hazards Regression Model for Mortality.
Variables with unadjusted p-values < 0.3 from univariable were included in the multivariable analysis.

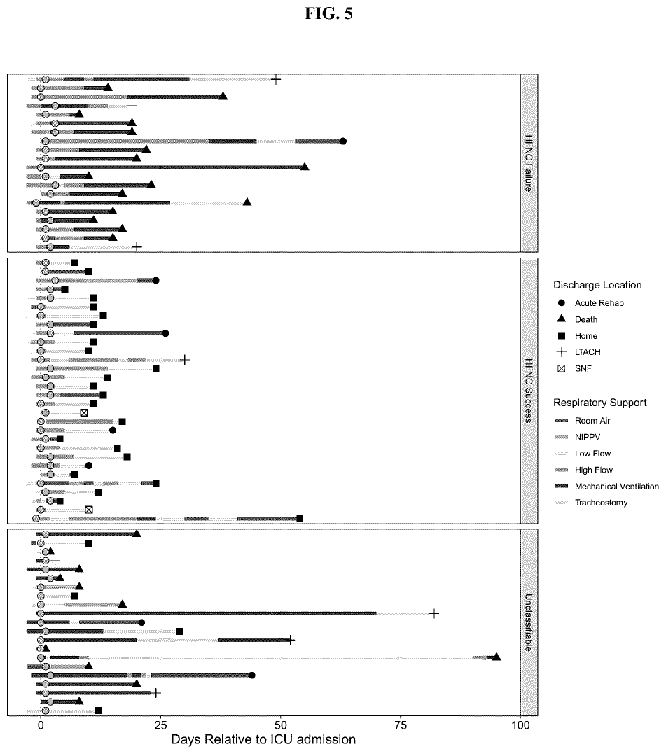

Although the 71 patients admitted to the ICU had severe respiratory compromise, a subset of 50 patients did not initially require mechanical ventilation and were treated with high-flow oxygen by nasal canula (HFNC) (FIG. 5). The course of respiratory failure in this group included 20 patients who progressed from HFNC to endotracheal intubation and 30 patients who de-escalated to low flow nasal cannula (LFNC). Patients in whom transitions could not be identified were most frequently already intubated at ICU admission or did not require respiratory support other than LFNC. Patient and microbiologic characteristics are stratified by progression of respiratory failure in Table 5. Variables found to be significant on univariable analysis were included in a multivariable logistic regression model which demonstrated higher MMP to be independently associated with progression of respiratory failure requiring intubation (RR=1.12, CI=1.04-1.21, p=0.005) (Table 6).

TABLE 5
Patient characteristics Stratified by Trajectory of Respiratory Failure

| | HFNC Success | | HFNC Failure | | |
|---|---|---|---|---|---|
| n | 30 | | 20 | | p |
| Baseline Characteristics | | | | | |
| Race (%) | | | | | 0.543 |
| Asian/Mideast Indian | 2 | (6.4) | 0 | (0.0) | |
| Black/African-American | 21 | (67.7) | 14 | (66.7) | |
| Hispanic | 2 | (6.4) | 3 | (14.3) | |
| White | 6 | (19.4) | 4 | (19.0) | |
| Male (%) | 35 | (48.4) | 12 | (60.0) | 0.685 |
| Age (median [IQR]) | 59.47 | [54.31, 67.43] | 67.96 | [59.23, 76.01] | 0.113 |
| Body Mass Index (median [IQR]) | 31.05 | [27.30, 38.80] | 30.66 | [26.58, 33.59] | 0.759 |
| Hypertension (%) | 20 | (60) | 12 | (66.7) | 0.857 |
| Hyperlipidema (%) | 8 | (26.7) | 10 | (45) | 0.3 |
| Diabetes (%) | 11 | (36.7) | 9 | (45.0) | 0.768 |
| Cancer (%) | 6 | (20) | 3 | (15) | 0.94 |
| Chronic Kidney Disease (%) | 1 | (3.3) | 4 | (20) | 0.149 |
| Clinical Characteristics | | | | | |
| SOFA Score (median [IQR]) | 4.00 | [4.0, 5.75] | 9.00 | [8.75, 9.00] | <0.001 |
| APACHE Score (median [IQR]) | 18.00 | [13.00, 20.50] | 21.00 | [17.00, 29.00] | 0.026 |
| Charison Comorbidity Index (median [IQR]) | 3.00 | [2.00, 4.75] | 4.00 | [2.00, 6.00} | 0.363 |
| Days From Symptom Onset (median [IQR]) | 5.00 | [3.00, 6.50] | 3.00 | [2.00, 8.00] | 0.905 |
| Admission (%) | | | | | 0.495 |
| ED | 22 | (73.3) | 12 | (60) | |
| Hospital Medicine | 6 | (20) | 7 | (35) | |
| OSH | 2 | (6.7) | 1 | (5.0) | |
| Medication Administration | | | | | |
| Antivirals | | | | | |
| Steroid Treatment (%) | 22 | (73.3) | 18 | (90.0) | 0.279 |
| Remdesivir Treatment (%) | 24 | (80.0) | 18 | (90.0) | 0.567 |
| Antibiotics | | | | | |
| Betalactams (%) | 5 | (16.7) | 4 | (20.0) | 1 |
| Levofloxicin (%) | 1 | (3.3) | 0 | (0.0) | 1 |
| Vancomycin (%) | 6 | (20.0) | 7 | (30.0) | 0.646 |
| Metronidazole (%) | 1 | (3.3) | 1 | (5.0) | 1 |
| Macrolides (%) | 7 | (23.3) | 7 | (35.0) | 0.563 |
| Doxycycline (%) | 4 | (13.3) | 0 | (0) | 0.242 |
| Trimethoprim-Sulfamethoxazole (%) | 4 | (13.3) | 2 | (10.0) | 1 |

Univariable analysis comparing patient characteristics and medication administration for high flow nasal cannula (HFNC) failure, defined as need for endotracheal intubation versus HFNC success, defined as transition to low flow nasal cannula. Categorical variables absolute number (percent within group). Categorical Variables compared with Chi-Square. Continuous variables were compared Wilcoxon Rank Sum Test. Adequate treatment with COVID-19 specific therapy included at least three consecutive days of therapy during index hospitalization. Sufficient total dose of dexamethasone was 18 mg and Remdesivir was 400 mg. Only antibiotics received 72 hours prior to fecal specimen collection are represented.

TABLE 6
Multivariable Regression Model on
Progression of Respiratory Failure

| Characterist | exp(Beta) | 95% CI¹ | p-value |
|---|---|---|---|
| Age | 1.00 | 1.00, 1.01 | 0.5 |
| Chronic Kidney Disease | 0.95 | 0.71, 1.29 | 0.8 |
| Steroid Treatment | 1.07 | 0.86, 1.34 | 0.6 |
| SOFA Score | 1.12 | 1.08, 1.17 | <0.001 |
| Doxycycline | 0.79 | 0.59, 1.06 | 0.13 |
| Microbiome Metabolic Profile | 1.12 | 1.04, 1.21 | 0.005 |

¹CI = Confidence Interval
Multivariable regression model for high flow nasal cannula failure.
Variables with p-values < 0.3 from univariable were included in the multivariable analysis.

### REFERENCES

The following references, some of which are cited above by number, and herein incorporated by reference in their entireties.
1. World Health Organization COVID-19 Dashboard. https://covid19.who.int/.
2. D′Agnillo, F. et al. Lung epithelial and endothelial damage, loss of tissue repair, inhibition of fibrinolysis, and cellular senescence in fatal COVID-19. Sci Transl Med 13, eabj7790 (2021).
3. Nienhold, R. et al. Two distinct immunopathological profiles in autopsy lungs of COVID-19. Nat Commun 11, 5086 (2020).
4. Channappanavar, R. et al. IFN-I response timing relative to virus replication determines MERS coronavirus infection outcomes. J Clin Invest 129, 3625-3639 (2019).
5. Xu, Z. et al. Pathological findings of COVID-19 associated with acute respiratory distress syndrome. Lancet Respir Medicine 8, 420-422 (2020).
6. Tay, M. Z., Poh, C. M., Rénia, L., MacAry, P. A. & Ng, L. F. P. The trinity of COVID-19: immunity, inflammation and intervention. Nat Rev Immunol 20, 363-374 (2020).
7. Layne, S. P., Walters, K.-A., Kash, J. C. & Taubenberger, J. K. More autopsy studies are needed to understand the pathogenesis of severe COVID-19. Nat Med 28, 427-428 (2022).
8. Abt, M. C. et al. Commensal Bacteria Calibrate the Activation Threshold of Innate Antiviral Immunity. Immunity 37, 158-170 (2012).
9. Ichinohe, T. et al. Microbiota regulates immune defense against respiratory tract influenza A virus infection. Proc National Acad Sci 108, 5354-5359 (2011).
10. Stefan, K. L., Kim, M. V., Iwasaki, A. & Kasper, D. L. Commensal Microbiota Modulation of Natural Resistance to Virus Infection. Cell 183, 1312-1324.e10 (2020).
11. Alwin, A. & Karst, S. M. The influence of microbiota-derived metabolites on viral infections. Curr Opin Virol 49, 151-156 (2021).
12. Belkaid, Y. & Hand, T. W. Role of the Microbiota in Immunity and Inflammation. Cell 157, 121-141 (2014).
13. Furusawa, Y. et al. Commensal microbe-derived butyrate induces the differentiation of colonic regulatory T cells. Nature 504, 446-450 (2013).
14. Smith, P. M. et al. The Microbial Metabolites, Short-Chain Fatty Acids, Regulate Colonic Treg Cell Homeostasis. Science 341, 569-573 (2013).
15. Arpaia, N. et al. Metabolites produced by commensal bacteria promote peripheral regulatory T cell generation. Nature 504, 451-455 (2013).
16. Campbell, C. et al. Bacterial metabolism of bile acids promotes generation of peripheral regulatory T cells. Nature 581, 475-479 (2020).
17. Li, W. et al. A bacterial bile acid metabolite modulates Treg activity through the nuclear hormone receptor NR4A1. Cell Host Microbe 29, 1366-1377.e9 (2021).
18. Steed, A. L. et al. The microbial metabolite desaminotyrosine protects from influenza through type I interferon. Science 357, 498-502 (2017).
19. Haak, B. W. et al. Impact of gut colonization with butyrate producing microbiota on respiratory viral infection following allo-HCT. Blood 131, blood-2018-01-828996 (2018).
20. Lee, J. R. et al. Butyrate-producing gut bacteria and viral infections in kidney transplant recipients: A pilot study. Transpl Infect Dis 21, e13180 (2019).
21. Mizutani, T. et al. Correlation Analysis between Gut Microbiota Alterations and the Cytokine Response in Patients with Coronavirus Disease during Hospitalization.

Microbiol Spectr 10, e01689-21 (2022).
22. Zuo, T. et al. Alterations in Gut Microbiota of Patients With COVID-19 During Time of Hospitalization. Gastroenterology 159, 944-955.e8 (2020).
23. Xu, X. et al. Integrated analysis of gut microbiome and host immune responses in COVID-19. Front Med-prc 16, 263-275 (2022).
24. Yeoh, Y. K. et al. Gut microbiota composition reflects disease severity and dysfunctional immune responses in patients with COVID-19. Gut 70, 698-706 (2021).
25. Moreira-Rosirio, A. et al. Gut Microbiota Diversity and C-Reactive Protein Are Predictors of Disease Severity in COVID-19 Patients. Front Microbiol 12, 705020 (2021).
26. Sokol, H. et al. SARS-CoV-2 infection in nonhuman primates alters the composition and functional activity of the gut microbiota. Gut Microbes 13, 1893113 (2021).
27. Paik, D. et al. Human gut bacteria produce TH17-modulating bile acid metabolites. Nature 603, 907-912 (2022).
28. Funabashi, M. et al. A metabolic pathway for bile acid dehydroxylation by the gut microbiome. Nature 582, 566-570 (2020).
29. Devlin, A. S. & Fischbach, M. A. A biosynthetic pathway for a prominent class of microbiota-derived bile acids. Nat Chem Biol 11, 685-690 (2015).
30. Sato, Y. et al. Novel bile acid biosynthetic pathways are enriched in the microbiome of centenarians. Nature 1-7 (2021) doi:10.1038/s41586-021-03832-5.
31. Galvin-Pena, S. et al. Profound Treg perturbations correlate with COVID-19 severity.

Biorxiv 2020.12.11.416180 (2020) doi:10.1101/2020.12.11.416180.
32. Bastard, P. et al. Autoantibodies against type I IFNs in patients with life-threatening COVID-19. Science 370, eabd4585 (2020).
33. Zhang, Q. et al. Inborn errors of type I IFN immunity in patients with life-threatening COVID-19. Science 370, eabd4570 (2020).
34. Schoefer, L., Mohan, R., Schwiertz, A., Braune, A. & Blaut, M. Anaerobic Degradation of Flavonoids by Clostridium orbiscindens. Appl Environ Microb 69, 5849-5854 (2003).
35. Dodd, D. et al. A gut bacterial pathway metabolizes aromatic amino acids into nine circulating metabolites. Nature 551, 648-652 (2017).
36. Bolger, A. M., Lohse, M. & Usadel, B. Trimmomatic: a flexible trimmer for Illumina sequence data. Bioinformatics 30, 2114-2120 (2014).
37. Li, D., Liu, C.-M., Luo, R., Sadakane, K. & Lam, T.-W. MEGAHIT: an ultra-fast single-node solution for large and complex metagenomics assembly via succinct de Bruijn graph. Bioinformatics 31, 1674-1676 (2015).
38. Seemann, T. Prokka: rapid prokaryotic genome annotation. Bioinformatics 30, 2068-2069 (2014).
39. Huerta-Cepas, J. et al. Fast Genome-Wide Functional Annotation through Orthology Assignment by eggNOG-Mapper. Mol Biol Evol 34, 2115-2122 (2017).
40. Kanehisa, M. & Goto, S. KEGG: Kyoto Encyclopedia of Genes and Genomes. Nucleic Acids Res 28, 27-30 (2000).
41. The Comprehensive Antibiotic Resistance Database. https://card.mcmaster.ca/.
42. Fluss, R., Faraggi, D. & Reiser, B. Estimation of the Youden Index and its Associated Cutoff Point. Biometrical J 47, 458-472 (2005).
43. Haak, B. W., Littmann, E. R., Chaubard, J.-L., Pickard, A. J., Fontana, E., Adhi, F., Gyaltshen, Y., Ling, L., Morjaria, S. M., Peled, J. U., van den Brink, M. R., Geyer, A. I., Cross, J. R., Pamer, E. G., & Taur, Y. (2018). Impact of gut colonization with butyrate-producing microbiota on respiratory viral infection following allo-HCT. Blood, 131(26), 2978-2986. https://doi.org/10.1182/blood-2018-01-828996.

1. A method of assessing the level of one or more biomarkers of mortality in the gut of the subject comprising:
(a) assessing a level of one or more bacterial species in a fecal sample from the subject; and/or
(b) assessing a level of one or more metabolomic biomarkers in the fecal sample from the subject.

2. The method of claim 1, wherein the subject suffers from respiratory insufficiency.

3. The method of claim 2, wherein the subject suffers from severe respiratory insufficiency.

4. The method of claim 1, wherein the subject suffers from COVID-19.

5. The method of claim 4, wherein the subject suffers from COVID-19 acute respiratory distress syndrome (ARDS).

6. The method of claim 1, wherein the subject is hospitalized.

7. The method of claim 6, wherein the subject is ventilated.

8. (canceled)

9. The method of claim 1, assessing one or more microbiomic biomarkers and one or more metabolomic biomarkers.

10. The method of claim 9, wherein one or more microbiomic biomarkers, selected from the level of one or more bacterial species belonging to the families Bacteroidaceae, Lachnospiraceae and Enterobacteriaceae or the phylum Proteobacteria, are assessed.

11. The method of claim 10, wherein an increased level of the one or more bacterial species belonging to the families Bacteroidaceae and Lachnospiraceae is associated with survival, a decreased level of the one or more bacterial species belonging to the families Bacteroidaceae and Lachnospiraceae is associated with mortality, a decreased level of the one or more bacterial species belonging to the family Enterobacteriaceae or phylum Proteobacteria is associated with survival, and an increased level of the one or more bacterial species belonging to the family Enterobacteriaceae or phylum Proteobacteria is associated with mortality.

12. (canceled)

13. The method of claim 1, wherein the level of one or more metabolomic biomarkers is assessed by mass spectrometric analysis of a sample from the subject.

14. The method of claim 1, wherein one or more metabolomic biomarkers, selected from the level of one or more of indole-3-carboxaldehyde, deaminotyrosine, deoxucholic acid, 3-oxodeoxycholic acid, 3-oxochenodeoxycholic acid, lithocholic acid, 3-aminoisobutyrate, serine, isodeoxycholic acid, toluate, and allolithocholic acid, are assessed.

15. The method of claim 14, wherein an increased level of one or more of indole-3-carboxaldehyde, deaminotyrosine, deoxucholic acid, 3-oxodeoxycholic acid, 3-oxochenodeoxycholic acid, lithocholic acid, 3-aminoisobutyrate, serine, and isodeoxycholic acid is associated with survival, a decreased level of one or more of indole-3-carboxaldehyde, deaminotyrosine, deoxucholic acid, 3-oxodeoxycholic acid, 3-oxochenodeoxycholic acid, lithocholic acid, 3-aminoisobutyrate, serine, and isodeoxycholic acid is associated with mortality, a decreased level of one or both of toluate and allolithocholic acid is associated with survival, and an increased level of one or both of toluate and allolithocholic acid is associated with mortality.

16. The method of claim 15, wherein the increased level is above a threshold level and the decreased level is below the threshold level.

17-18. (canceled)

19. The method of claim 1, wherein the level of two or more of deoxycholic acid, isodeoxycholic acid, lithocholic acid, and desaminotyrosine are assessed, wherein high levels of the two or more of deoxycholic acid, isodeoxycholic acid, lithocholic acid, and desaminotyrosin is associated with a high risk of mortality.

20. The method of claim 19, wherein high levels are levels above a threshold value.

21. The method of claim 20, wherein the threshold is between 70 μM and 110 μM for deoxycholic acid, between 0.8 μM and 1.2 μM for isodeoxycholic acid, between 220 μM and 300 μM for lithocholic acid, and between 10 μM and 30 μM for desaminotyrosin.

22. The method of claim 21, wherein the threshold is about 90 μM for deoxycholic acid, about 1 μM for isodeoxycholic acid, about 258 μM for lithocholic acid, and about 21 μM for desaminotyrosin.

23. The method of claim 19, comprising assessing the levels of each of deoxycholic acid, isodeoxycholic acid, lithocholic acid, and desaminotyrosine.

24. A method of treating a subject suffering from COVID-19 acute respiratory distress syndrome (ARDS), comprising assessing the subject's risk of mortality by the method of claim 1, wherein if the subject is determined to be at increased risk of mortality fecal augmentation therapy is administered, and wherein if the subject is determined to be at decreased risk of mortality fecal augmentation therapy is not administered.
